(19) United States
(12) Patent Application Publication
(10) Pub. No.: US 2007/0281028 A1
(43) Pub. Date: Dec. 6, 2007

(54) Drug Delivery of a Cox Inhibitor from Embolic Agents

(75) Inventors: Andrew Lennard Lewis, Surrey (GB); Peter William Stratford, Surrey (GB); Maria Victoria Gonzalez Fajardo, Surrey (GB); Hind Hassan Sid Ahmed Goreish, Surrey (GB)

Correspondence Address: SUGHRUE MION, PLLC, 2100 PENNSYLVANIA AVENUE, N.W., SUITE 800, WASHINGTON, DC, 20037, US

(73) Assignee: Biocompatibles Uk Limited, Surrey (GB)

(21) Appl. No.: 11/573,172

(22) PCT Filed: Aug. 4, 2005
(86) PCT No.: PCT/GB05/03065

§ 371 Date: Jul. 11, 2007

(30) Foreign Application Priority Data
Aug. 4, 2004 (EP) 04254665.5

(51) Int. Cl. A61K 31/415; A61K 31/19; A61K 31/192; A61K 31/196; A61K 31/42; A61K 45/00; A61P 35/00; A61K 9/00; A61K 31/54; A61K 31/21; A61K 31/341; A61K 31/40

(52) U.S. Cl. 424/487; 424/484; 424/486; 514/226.5; 514/374; 514/406; 514/411; 514/413; 514/473; 514/510; 514/557; 514/567; 514/568; 514/569; 514/789

(57) ABSTRACT

A pharmaceutical composition for malignant tumour embolisation comprises a polymer and, associated with the polymer in a releasable form, a COX inhibitor, e.g. a non-steroidal anti inflammatory drug, such as ibuprofen. The polymer is preferably-in particulate form, such as in the form of microspheres. A suitable polymer is a crosslinked polyvinyl alcohol polymer formed by the copolymerisation of PVA macromer with other ethylenically unsaturated monomers. The composition provides a synergistic treatment for the symptoms of malignant tumours, leading to tumour necrosis or ischaemia, with anti-angiogenic effects, promotion of apoptosis, decrease in invasiveness of tumour cells and resultant tumour regression.

The present invention relates to compositions which embolise malignant tumours including metastases and deliver drugs at the site of embolisation. The drugs have cyclooxygenase (COX) inhibitory properties.

Embolisation therapy involves the introduction of an agent into the vasculature in order to bring about the deliberate blockage of a particular vessel. This type of therapy is particularly useful for blocking abnormal connections between arteries and veins (such as arteriovenous malformations, or AVMs), and also for occluding vessels that feed certain hyper-vascularised tumours, in order to starve the abnormal tissue and bring about its necrosis and shrinkage.

The process of embolisation may induce tumour necrosis or ischemia depending upon the extent of the embolisation. The response of the tumour cells to the hypoxic environment can result in an ensuing angiogenesis in which new blood vessels are grown to compensate for the loss of flow to the tumour by the embolisation. It would be desirable, therefore, to combine embolisation with the administration of agents that could prevent the ensuing angiogenic response.

Prostaglandins (PGs) have diverse biological functions in the body and distinct receptors for the different types of PGs that mediate their action. PGs are formed from unsaturated fatty acids by the action of cyclooxygenases (COX). Two COX enzymes that have been identified are COX-1, which has a house-keeping function, and COX-2, the production of which is highly regulated. It is induced in reproductive tissues during ovulation, implantation and labour, in inflammatory cells including those associated with arthritis, and in tumour cells by cytokines and tumour promoters. COX-2 expression has also been detected in the brain, kidney and in some cells in other organs, the function of which in these locations is not well understood.

Recent studies have shown that the levels of COX-2 are elevated in certain types of cancers like colorectal, lung, breast and liver. COX-2 is reported to be expressed in 80-90% of colorectal cancer cells. Epidemiological studies as early as 1991, showed that regular use of aspirin or other traditional NSAIDs might reduce the risk of death from colon cancer (Thun M. J.; Namboodiri, M. M.; Heath, C. W. New England Journal of Medicine 1991, 325, 1593-1596). These and other similar observations lead to the hypothesis that COX-2 and certain prostaglandins might play crucial role in carcinogenesis and future use of conventional non-steroidal anti-inflammatory drugs (NSAIDs) for treatment of cancer is under investigation.

The mechanisms by which COX-2 attributes to cancer development are proposed to be via enhancing angiogenesis, inhibition of apoptosis, increase in invasiveness of tumour cell and increased cellular adhesion. Angiogenesis is important feature of inflammation and cancer growth and metastasis. These effects are mostly brought by prostaglandins which are produced by the action of COX-2:

James Liebmann (Cancer Investigation, 22(2), 324-325, 2004) discusses why COX-2 inhibitors combined with other drugs as a cocktail may act to block several pathways in tumourigenesis and provide more successful therapies. He references the pre-clinical work of Blanke (Cancer Invest 2002) in telling us why COX-2 inhibitors should work. Moreover, Haller (Seminars in Oncology, 30(4), 2-8, 2003) describes their use in oncology and outlines a schematic to demonstrate how COX-2 is involved in not only early stages of cancer but the progression to advanced disease and metastasis: (See FIG. 21, the Role of COX-2 inhibition in controlling tumourigenesis).

COX-2 is expressed within human tumour neovasculature as well as in neoplastic cells present in human colon, breast, prostate, and lung cancer biopsy tissue [Masferrer J L, Leahy K M, Koki A T, Zweifel B S, Settle S L, Woerner B M, Edwards D A, Flickinger A G, Moore R J, Seibert K. Cancer Res. 2000 Mar. 1;60(5):1306-11]. COX-2 enhances angiogenesis of the tumour cell, as VEGF synthesis is up-regulated by PGE2, the product of COX-2 action. Hence COX-2 contributes to the production of pro-angiogenic factors, including VEGF, the migration of endothelial cells through collagen matrices and the formation of capillary networks in vitro. Indeed it has been shown that NS-398, a COX-2 inhibitor, diminished the expression of these factors in colorectal cancer cell line (Tsujii, M. et al. Am. J. Phsyiol. 1998 274 (6Pt1), G1061-7.

COX-1 can also contribute to angiogenesis as non-selective NSAIDs have decreased vascularization of xenograft not expressing COX-2. NSAID also inhibited tubule formation even when cells do not express COX-2 (Tsujii, 1998 et al op.cit, Jones, et al. Nat. Med. 1999 Dec. 5(12):1418-23).

Although ibuprofen may have some influence as an anti-angiogenic factor, it is not normally considered to be a classic anti-angiogenic agent. Classic anti-angiogenic agents include tyrosine kinase inhibitors such as avastatin, ZD6474 and semaxanib or potent angiostatic agents like fumagillin and TNP-470.

COX-2 over-expression leads to phenotypic changes involving increased adhesion to extracellular matrix and inhibition of apoptosis in intestinal epithelial cells that could enhance their tumorigenic potential, COX inhibitors have been shown to reverse these changes, (Tsujii M, DuBois R N. Cell 83:493-501, 1995). Furthermore over-expression of COX-2 has been shown to inhibit apoptosis in intestinal mucosa (Sheng H, Shao J, Morrow J D, Beauchamp R D, DuBois R N Cancer Res. 1998 Jan. 15;58(2):362-6). This may be a consequence of the production of PGE2, which may send improper signals in the cells thereby stimulating inappropriate cell growth or reducing apoptosis [Sheng H, et al., op.cit.

Increase in the production of matrix metalloproteinases (MMPs) has been linked to COX-2. Tsujii M, Kawano S, et al. Proc.Natl.Acad. Sci. USA 94:3336-3340, 1997).

COX-2 was also shown to affect MMP activity and increases collagenase levels, thus increasing tumour cell invasiveness.

Increased survival of tumour cells has been linked to change in cellular adhesion to ECM as a result of over expression of COX-2 (Tsujii M, et al. 1995 op.cit.).

Hypoxia-inducible factors (HIF-1 alpha and HIF-2 alpha) are considered potential targets for anti-neoplastic therapy because they regulate the expression of genes that contribute to tumour cell survival, aggressiveness, and angiogenesis. Non-specific NSAIDs like ibuprofen, diclofenac and keterolac inhibited both HIF-1 alpha and HIF-2 alpha gene expression compared to the inhibition of HIF-2 alpha only by the COX-2 selective NS-398 HIFs inhibition by NSAID was COX-2 independent [Palayoor ST, et al. 2003 ;9(8):3150-3157].

NSAIDs are medications which, as well as having pain-relieving (analgesic) effects, have the effect of reducing inflammation when used over a period of time. A new class of NSAIDs, cyclooxygenase-2 (COX-2) inhibitors, selectively inhibits inflammatory prostaglandins (PGs). These new drugs have a lower complication rate and do not tend to produce ulcers. There are many different types of NSAIDs, including aspirin and other salicylates. Examples include; ibuprofen (Motrin, Advil), naproxen (Naprosyn), diclofenac (Voltaren), ketoprofen (Orudis), indomethacin (Indocin), and newer ones such as celecoxib (Celebrex), the first COX-2 inhibitor on the market, and rofecoxib (Vioxx), which was recently released:

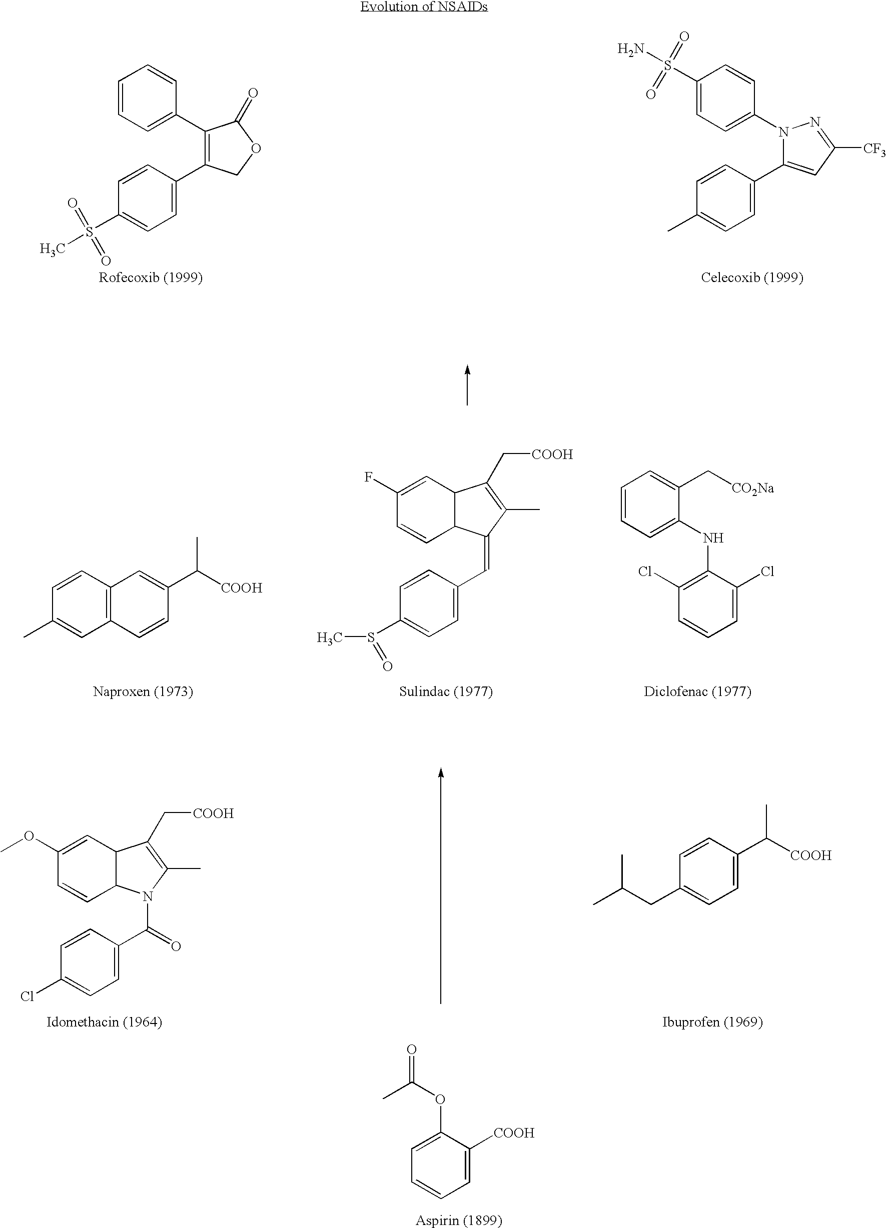

The primary mechanism of action in NSAIDs is by interfering with the cyclooxygenase pathway (enzymes that make prostaglandins) and a resultant decrease in prostaglandin synthesis.

Inhibitors of COX have activities against both enzymes but many are selective to one or other of the enzymes.

Inhibitors with high COX-1 selectivity are found to have undesirable side effects on the gastro intestinal tract, manifest when delivered orally. The recently launched COX-2 selective inhibitors reduce such side effects when administered orally.

In WO-A-0168720, PVA based compositions for embolotherapy are described. The PVA is, initially, derivatised to form a macromonomer, having pendant acrylic groups. Subsequently, these acrylic groups are polymerised, optionally in the presence of comonomer, to form a water-insoluble water-swellable polymer matrix. The polymerisation reaction may be carried out in situ, whereby the PVA is rendered water-insoluble after delivery into the vessel, at the embolisation site. Alternatively, the polymerisation is conducted prior to delivery, generally to form microspheres, which are delivered in suspension in an aqueous vehicle.

In WO-A-0168720, it is suggested that biologically active agents may be included in the embolic compositions, whereby active agent may be delivered from the formed hydrogel. One class of active agents is chemotherapeutic agents. Examples of chemotherapeutic agents are cisplatin, doxorubicin and mitomycin and lipiodol. The compositions may be used to embolise tumours such as liver tumours.

WO-A-0023054 describes cross-linked polyvinyl alcohol microspheres for use as embolic agents. The compositions may also comprise anti-angiogenic agents. Examples of anti-angiogenic agents include the classic anti-angiogenic agents and many other actives, but there are no data or hypotheses to support the assertions that the listed compounds are in fact anti-angiogenic. Included in the list are ibuprofen and indomethacin. Indications that may be treated by the compositions include primary colorectal cancer, hepatocellular carcinomas, liver metastases, bone metastases, cancers of the head and neck, intercranial meningiomas and melanomas. The polymer particles may contain collagen, ionic dextran derivatives and/or imaging agents. The only worked example which includes a specifically named chemotherapeutic agent discloses loading of polyvinylalcohol microspheres with thalidomide. It is suggested that particles coated with cationic dextran derivatives may be useful to adsorb anti-angiogenic or anti-inflammatory agents by an ion-exchange process.

Gohel, M. C. et al, in Drug Development and Industrial Pharmacy 25(2), 247-251 (1999) describe drug delivery systems for controlled release of diclofenac sodium, comprising microspheres of cross-linked polyvinyl alcohol. In the loading method, PVA is cross-linked using glutaraldehyde in the presence of the drug. The microspheres have sizes in the range 355 to 500 μm. There is no disclosure of how the product was intended to be delivered nor the indications to be treated. However in the dissolution experiments the microspheres are placed in gelatin capsules, perhaps simulating an orally administrable dosage form.

Our co-pending application number PCT/GB04/00698 discloses the use of compositions comprising COX inhibitors absorbed in polymeric embolic agents in the treatment of uterine fibroids, which are benign tumours, i.e. non-malignant.

According to the present invention there is provided a new use of water-insoluble polymer and, associated with polymer in a releasable form, a pharmaceutically active agent which is a COX inhibitor, in the manufacture of a composition for use in a method of malignant tumour embolisation, in which the pharmaceutical active is released from the polymer at the site of embolisation.

The polymer is a water-insoluble material. Although it may be biodegradable, so that drug may be released substantially by erosion of polymer matrix to release drug from the surface, preferably the polymer is substantially biostable.

It is preferred for the polymer to be water-swellable. Water-swellable polymer useful in the invention preferably has a equilibrium water content, when swollen in water at 37° C., measured by gravimetric analysis, in the range of 40 to 99 wt %, preferably 75 to 95%.

The polymer may be in the form of a coating on an embolic device such as a metal coil. Preferably, however, the embolic agent is in the form of particles of bulk polymer, or alternatively foamed polymer, having open or closed cells therein. Alternatively, the polymeric agent may be formed in situ, by delivery of a liquid agent and curing at the site of embolisation to form an insoluble polymer matrix.

In the preferred embodiment of the invention, the composition which is administered to a patient in need of embolisation therapy, is in the form of a suspension of particles of water-swollen water-insoluble polymer. Preferably the particles are graded into calibrated size ranges for accurate embolisation of vessels. The particles preferably have sizes when equilibrated in water at 37° C., in the range 40 to 1500 μm, more preferably in the range 100 to 1200 μm. The calibrated ranges may comprise particles having diameters with a bandwidth of about 100 to 300 μm. The size ranges may be for instance 100 to 300 μm, 300 to 500 μm, 500 to 700 μm, 700 to 900 μm and 900 to 1200 μm. Preferably the particles are substantially spherical in shape. Such particles are referred to herein as microspheres.

Generally the polymer is covalently crosslinked, although it may be appropriate for the polymer to be ionically crosslinked, at least in part. Although it may be suitable to use polymers which are derived from natural sources, such as albumin, alginate, gelatin, starch, chitosan or collagen, all of which have been used as embolic agents the polymer is preferably substantially free of naturally occurring polymer or derivatives. It is preferably formed by polymerising ethylenically unsaturated monomers in the presence of di- or higher-functional crosslinking monomers. The ethylenically unsaturated monomers may include an ionic (including zwitterionic) monomer.

Copolymers of hydroxyethyl methacrylate, acrylic acid and crosslinking monomer, such as ethylene glycol dimethacrylate or methylene bisacrylamide, as used for etafilcon A based contact lenses may be used.

Copolymers of N-acryloyl-2-amino-2-hydroxymethyl-propane-1,3-diol and N,N-bisacrylamide may also be used.

Other polymers are cross-linking styrenic polymers e.g. with ionic substituents, of the type used as separation media or as ion exchange media.

Another type of polymer which may be used to form the water-swellable water-insoluble matrix is polyvinyl alcohol crosslinked using aldehyde type crosslinking agents such as glutaraldehyde. For such products, the polyvinyl alcohol (PVA) may be rendered ionic or may be substantially non-ionic. For instance the PVA may be rendered ionic by providing pendant ionic groups by reacting a functional ionic group containing compound with the hydroxyl groups. Examples of suitable functional groups for reaction with the hydroxyl groups are acylating agents, such as carboxylic acids or derivatives thereof, or other acidic groups which may form esters. Suitable commercially available embolic agents based on polyvinyl alcohol which may be used in the invention are Ivalon, Trufill and Contour SE (trade marks).

The invention is of particular value where the polymer matrix is formed of a polyvinyl alcohol macromer, having more than one ethylenically unsaturated pendant group per molecule, by radical polymerisation of the ethylenic groups. Preferably the PVA macromer is copolymerised with ethylenically unsaturated monomers for instance including a nonionic and/or ionic monomer.

The PVA macromer may be formed, for instance, by providing PVA polymer, of a suitable molecular weight such as in the range 1000 to 500,000 D, preferably 10,000 to 100,000 D, with pendant vinylic or acrylic groups. Pendant acrylic groups may be provided, for instance, by reacting acrylic or methacrylic acid with PVA to form ester linkages through some of the hydroxyl groups. Other methods for attaching vinylic groups capable of polymerisation onto polyvinyl alcohol are described in, for instance, U.S. Pat. No. 4,978,713 and, preferably, U.S. Pat. Nos. 5,508,317 and 5,583,163. Thus the preferred macromer comprises a backbone of polyvinyl alcohol to which is linked, via a cyclic acetal linkage, an (alk)acrylaminoalkyl moiety. Example 1 describes the synthesis of an example of such a macromer known by the approved named nelfilcon B. Preferably the PVA macromers have about 2 to 20 pendant ethylenic groups per molecule, for instance 5 to 10.

Where PVA macromers are copolymerised with ethylenically unsaturated monomers including an ionic monomer, the ionic monomer preferably has the general formula I
Y¹BQ
in which Y¹ is selected from 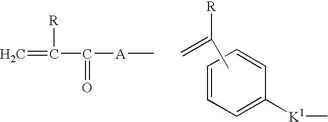
CH₂═C(R)—CH₂—O—, CH₂═C(R)—CH₂ OC(O)—, CH₂═C(R)OC(O)—, CH₂═C(R)—O—, CH₂═C(R)CH₂OC(O)N(R¹)—, R²OOCCR═CRC(O)—O—, RCH═CHC(O)O—, RCH═C(COOR²)CH₂—C(O)—O—, 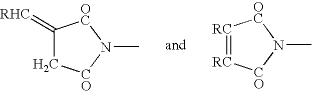
wherein:

R is hydrogen or a C₁-C₄ alkyl group;

R¹ is hydrogen or a C₁-C₄ alkyl group;

R² is hydrogen or a C₁₋₄ alkyl group or BQ where B and Q are as defined below;

A is —O— or —NR¹—;

K¹ is a group —(CH₂)ᵣOC(O)—, —(CH₂)ᵣC(O)O—, —(CH₂)ᵣOC(O)O—, —(CH₂)ᵣNR³—, —(CH₂)ᵣNR³C(O)—, —(CH₂)ᵣC(O)NR³—, —(CH₂)ᵣNR³C(O)O—, —(CH₂)ᵣOC(O)NR³, —(CH₂)ᵣNR³C(O)NR³— (in which the groups R³ are the same or different), —(CH₂)ᵣO—, —(CH₂)ᵣSO₃—, or, optionally in combination with B¹, a valence bond and r is from 1 to 12 and R³ is hydrogen or a C₁-C₄ alkyl group;

B is a straight or branched alkanediyl, oxaalkylene, alkanediyloxaalkanediyl, or alkanediyloligo(oxaalkanediyl) chain optionally containing one or more fluorine atoms up to and including perfluorinated chains or, if Q or Y¹ contains a terminal carbon atom bonded to B a valence bond; and

Q is an ionic group.

An anionic group Q may be, for instance, a carboxylate, carbonate, sulphonate, sulphate, nitrate, phosphonate or phosphate group. The monomer may be polymerised as the free acid or in salt form. Preferably the pKₐ of the conjugate acid is less than 5.

A suitable cationic group Q is preferably a group N⁺R₃⁴, P⁺R₃ R⁵ or S⁺R₂ R⁵

in which the groups R⁴ are the same or different and are each hydrogen, C₁₋₄-alkyl or aryl (preferably phenyl) or two of the groups R⁴ together with the heteroatom to which they are attached from a saturated or unsaturated heterocyclic ring containing from 5 to 7 atoms the groups R⁵ are each OR⁴ or R⁴. Preferably the cationic group is permanently cationic, that is each R⁴ is other than hydrogen. Preferably a cationic group Q is N⁺R⁴₃ in is which each R⁴ is C₁₋₄-alkyl, preferably methyl.

A zwitterionic group Q may have an overall charge, for instance by having a divalent centre of anionic charge and monovalent centre of cationic charge or vice-versa or by having two centres of cationic charge and one centre of anionic charge or vice-versa. Preferably, however, the zwitterion has no overall charge and most preferably has a centre of monovalent cationic charge and a centre of monovalent anionic charge.

Examples of zwitterionic groups which may be used as Q in the present invention are disclosed in WO-A-0029481.

Where the ethylenically unsaturated monomer includes zwitterionic monomer, for instance, this may increase the hydrophilicity, lubricity, biocompatibility and/or haemocompatibility of the particles. Suitable zwitterionic monomers are described in our earlier publications WO-A-9207885, WO-A-9416748, WO-A-9416749 and WO-A-9520407. Preferably a zwitterionic monomer is 2-methacryloyloxy-2′-trimethylammonium ethyl phosphate inner salt (MPC).

In the monomer of general formula I preferably Y¹ is a group CH₂═CRCOA- in which R is H or methyl, preferably methyl, and in which A is preferably NH. B is preferably an alkanediyl group of 1 to 12, preferably 2 to 6 carbon atoms. Such monomers are acrylic monomers.

There may be included in the ethylenically unsaturated monomer diluent monomer, for instance non-ionic monomer. Such a monomer may be useful to control the pKₐ of the acid groups, to control the hydrophilicity or hydrophobicity of the product, to provide hydrophobic regions in the polymer, or merely to act as inert diluent. Examples of non-ionic diluent monomer are, for instance, alkyl (alk) acrylates and (alk) acrylamides, especially such compounds having alkyl groups with 1 to 12 carbon atoms, hydroxy, and di-hydroxy-substituted alkyl(alk) acrylates and -(alk) acrylamides, vinyl lactams, styrene and other aromatic monomers.

In the polymer matrix, where there is ionic group present the level of ion is preferably in the range 0.1 to 10 meq g⁻¹, preferably at least 1.0 meq g⁻¹.

Where PVA macromer is copolymerised with other ethylenically unsaturated monomers, the weight ratio of PVA macromer to other monomer is preferably in the range of 50:1 to 1:5, more preferably in the range 20:1 to 1:2. In the ethylenically unsaturated monomer the ionic monomer is preferably present in an amount in the range 10 to 100 mole %, preferably at least 25 mole %.

The polymer may be formed into particles in several ways. For instance, the crosslinked polymer may be made as a bulk material, for instance in the form of a sheet or a block, and subsequently be comminuted to the desired size. Alternatively, the crosslinked polymer may be formed as such in particulate form, for instance by polymerising in droplets of monomer in a dispersed phase in a continuous immiscible carrier. Examples of suitable water-in-oil polymerisations to produce particles having the desired size, when swollen, are known. For instance U.S. Pat. No. 4,224,427 describes processes for forming uniform spherical beads (microspheres) of up to 5 mm in diameter, by dispersing water-soluble monomers into a continuous solvent phase, in a presence of suspending agents. Stabilisers and surfactants may be present to provide control over the size of the dispersed phase particles. After polymerisation, the crosslinked microspheres are recovered by known means, and washed and optionally sterilised. Preferably the particles eg microspheres, are swollen in an aqueous liquid, and classified according to their size.

Examples of specific active agents which are COX inhibitors that are useful in the present invention are:

celecoxib (Celebrex)

rofecoxib (Vioxx)

diclofenac (Voltaren, Cataflam)

diflunisal (Dolobid)

etodolac (Lodine)

flurbiprofen (Ansaid)

ibuprofen (Motrin, Advil)

indomethacin (Indocin)

ketoprofen (Orudis, Oruvail)

ketorolac (Toradol)

nabumetone (Relafen)

naproxen (Naprosyn, Alleve)

oxaprozin (Daypro)

piroxicam (Feldene)

sulindac (Clinoril)

tolmetin (Tolectin)

The active agent may be selective for COX-1. The invention allows local delivery of the active to the site of embolisation, and the target tumours. This avoids systemic delivery and the associated side effects described above with such actives, exhibited especially when the active is administered orally.

The active may be COX-2 selective.

The combination of tumour necrosis or ischemia induced by the embolic agent and anti-angiogenic effect of the COX inhibitor which is expected to follow should avoid angiogenesis which might otherwise ensue from the hypoxic environment created by embolisation. The composition used in the invention is expected to lead to a reduction in angiogenesis, promotion of apoptosis and decreased invasiveness of tumour cells. This is expected to lead to tumour regression. The invention is expected to be of benefit in the treatment of primary and secondary tumours which are hypervascular and hence embolisable, such as primary liver cancer (hepatocellular carcinoma, HCC), metastases to the liver (colorectal, breast, endocrine), and renal, bone, breast and lung tumours.

Suitable COX selective inhibitors are shown in the following table:

| Log [IC₈₀ ratio WHMA COX-2/COX-1)] | Drugs |
|---|---|
| −2 to −1 | DFP |
| | L-745337 |
| | Rofecoxib |
| | NS398 |
| | Etodolac |
| −1 to 0 | Meloxicam |
| | Celecoxib |
| | Nimesulide |
| | Diclofenac |
| | Sulindac Sulphide |
| | Meclofenamate |
| | Tomoxiprol |
| | Piroxicam |
| | Diflunisal |
| | Sodium Salicylate |
| 0 | Niflumic Acid |
| | Zomepirac |
| | Fenoprofen |
| 0 to 1 | Amypyrone |
| | Ibuprofen |
| | Tolmetin |
| | Naproxen |
| | Aspirin |
| | Indomethacin |
| | Ketoprofen |
| 1 to 2 | Suprofen |
| | Flurbiprofen |
| 2 to 3 | Ketorolac |

WHMA=William Harvey Human Modified Whole Blood Assay

The table refers to the Log [IC₈₀ ratio WHMA COX-2/COX-1)] for the agents which have been assayed by William Harvey Human Modified Whole Blood Assay. Those drugs with a “0” value indicate equal potency, i.e. an IC₈₀ ratio of 1. Values above “0” indicates the drug is more selective to COX-1 and values below “0” indicates the drug is more selective to COX-2.

DFP is diisopropylphosphofluoridate

L-745337 is 5-methanesulphonamide-6-(2,4-difluorothiophenyl)-1-indanone.

Values from Warner T. D. et al, Proc. Natl. Acad. Sci (1999) 96, 7563.

The pharmaceutical agent is associated with the polymer preferably so as to allow controlled release of the agent over a period. This period may be from several hours to weeks, preferably at least up to a few days, preferably up to 72 hours. The agent may be electrostatically, or covalently bonded to the polymer or held by Van der Waal's interactions. Since many COX inhibitors are acids, increased loading levels and slower release rates may be achievable where the polymer is cationic.

The pharmaceutical active may be incorporated into the polymer matrix by a variety of techniques. In one method, the active may be mixed with a precursor of the polymer, for instance a monomer or macromer mixture or a cross-linkable polymer and cross-linker mixture, prior to polymerising or crosslinking. Alternatively, the active may be loaded into the polymer after it has been crosslinked. For instance, particulate dried polymer may be swollen in a solution of active, preferably in water or in an alcohol such as ethanol, optionally with subsequent removal of non-absorbed agent and/or evaporation of solvent. A solution of the active, in an organic solvent such as an alcohol, or, more preferably, in water, may be sprayed onto a moving bed of particles, whereby drug is absorbed into the body of the particles with simultaneous solvent removal. Most conveniently, we have found that it is possible merely to contact swollen particles suspended in a continuous liquid vehicle, such as water, with an aqueous alcoholic solution of drug, over a period, whereby drug becomes absorbed into the body of the particles. Techniques to fix the drug in the particles may increase loading levels, for instance, precipitation by shifting the pH of the loading suspension to a value at which the active is in a relatively insoluble form. The swelling vehicle may subsequently be removed or, conveniently, may be retained with the particles as part of the product for subsequent use as an embolic agent or the swollen particles may be used in swollen form in the form of a slurry, i.e. without any or much liquid outside the swollen particles. Alternatively, the suspension of particles can be removed from any remaining drug loading solution and the particles dried by any of the classical techniques employed to dry pharmaceutical-based products. This could include, but is not limited to, air drying at room or elevated temperatures or under reduced pressure or vacuum; classical freeze-drying; atmospheric pressure-freeze drying; solution enhanced dispersion of supercritical fluids (SEDS). Alternatively the drug-loaded microspheres may be dehydrated using an organic solvent to replace water in a series of steps, followed by evaporation of the more volatile organic solvent. A solvent should be selected which is a non-solvent for the drug.

In brief, a typical classical freeze-drying process might proceed as follows: the sample is aliquoted into partially stoppered glass vials, which are placed on a cooled, temperature controlled shelf within the freeze dryer. The shelf temperature is reduced and the sample is frozen to a uniform, defined temperature. After complete freezing, the pressure in the dryer is lowered to a defined pressure to initiate primary drying. During the primary drying, water vapour is progressively removed from the frozen mass by sublimation whilst the shelf temperature is controlled at a constant, low temperature. Secondary drying is initiated by increasing the shelf temperature and reducing the chamber pressure further so that water absorbed to the semi-dried mass can be removed until the residual water content decreases to the desired level. The vials can be sealed, in situ, under a protective atmosphere if required.

Atmospheric pressure freeze-drying is accomplished by rapidly circulating very dry air over a frozen product. In comparison with the classical freeze-drying process, freeze-drying without a vacuum has a number of advantages. The circulating dry gas provides improved heat and mass transfer from the frozen sample, in the same way as washing dries quicker on a windy day. Most work in this area is concerned with food production, and it has been observed that there is an increased retention of volatile aromatic compounds, the potential benefits of this to the drying of biologicals is yet to be determined. Of particular interest is the fact that by using atmospheric spray-drying processes, instead of a cake, a fine, free-flowing powder is obtained. Particles can be obtained which have submicron diameters, this is ten-fold smaller than can be generally obtained by milling. The particulate nature, with its high surface area results in an easily rehydratable product, currently the fine control over particle size required for inhalable and transdermal applications is not possible, however there is potential in this area.

A preferred method of loading an active which has an acid group into a water-insoluble, water-swellable polymer vehicle includes the steps of

a) contacting water-swellable water-insoluble polymer with an aqueous solution of the agent at a pH at above the pKa of the acid group of the agent,

b) adding an acid to the product of step a) so as to reduce the pH of the aqueous liquid in contact with polymer to below the pKa of the acid group of the active; and

c) recovering the polymer with loaded agent in free acid form.

This method is of value for the COX inhibitors mentioned above whose free acid form, which is to be the form of the administered compound, is relatively water-insoluble. Such compounds include napoxen, sulindac, diclofenac, indomethacin, ibuprofen, acetyl salicylate, ketorolac, ketoprofen, flurbiprofen and suprofen, preferably ibuprofen.

Preferably the pH of the aqueous solution in step a) is at least 5, and the pH of the liquid after step b) is less than 3, as the acid group is a carboxylic acid in all these compounds.

Although the composition may be made up from polymer and COX-inhibitor immediately before administration, it is preferred that the composition is preformed. Dried polymer-COX inhibitor particles may be hydrated immediately before use. Alternatively the composition which is supplied may be fully compounded and preferably comprises polymer particles with adsorbed or absorbed COX inhibitor, imbibed water e.g. physiological saline and extra particulate liquid e.g. saline.

The level of COX inhibitor in the composition which is administered is preferably in the range 0.1 to 1000 mg/ml composition, preferably 10 to 100 mg/ml. Preferably the chemoembolisation method is repeated 1 to 5 times and for each dose the amount of COX inhibitor administered is in the range 0.1 to 1000 mg/ml, preferably 10 to 100 mg/ml. Based on the release data shown in the examples below, we believe this will give therapeutically effective concentrations in the blood vessels at a tumour and that significant levels of intracellular delivery should take place whereby a therapeutic effect will be achieved. The adverse side-effects of systemic COX inhibitors and/or of COX inhibitors on the GI tract will be avoided.

Oral doses of COX inhibitors are absorbed into the blood stream whereby at least 99%+of the drug becomes bound to plasma proteins such as albumin and is inactive. Of the remaining active drug, this will be distributed around the body where some may act upon the specific target which is responsible for the inflammation. This demonstrates the potency of such drugs. Hence, local delivery from the embolic agent directly into the tissue where the inflammatory reaction is likely to be induced will greatly enhance targeting. As the drug diffuses directly through vessel walls and into the surrounding tissues there may be a lower propensity for inactivation by binding to plasma protein, which could further enhance efficacy. A study by Fernandez-Carballido et al (Int J Pharmaceutics, 279, 33-41, 2004) was addressing the local delivery of ibuprofen-loaded microspheres into joints to treat rheumatoid arthritis. Based on a volume of 10 ml of synovial fluid and a transfer rate constant from synovial fluid to plasma of 0.3 h⁻¹, they calculated that a therapeutic dose would be achieved in the intraarticular cavity if the ibuprofen concentration could be kept at 24 μg/h. With doses of ibuprofen in the region 1-100 mg per gram of wet microspheres (per ml composition administered) and the insolubility of the drug in aqueous media, it could be expected that release rates exceeding 24 μg/h for prolonged periods could be sustained in-vivo from microspheres of the present invention. Shoen et al. (J Biomed.Mater.Res., 20(6), 709-21, 1986) used a mouse lung model to assess the pulmonary reaction to IV injected divinylbenzene copolymer beads (30-70 micron size) that were used to embolise the lung. 5 mg/kg and 25 mg/kg of indomethacin, 5 mg/kg of ibuprofen and 5 mg/kg of aspirin were prepared in sterile water and at a dilution to allow a 1 ml doe to be injected intraperitoneally immediately post embolisation and 24 h later. Even with this less elegant delivery method, they observed that the NSAIDs significantly reduced tissue reaction (measured as granuloma area) and also the volume of inflammatory exudate by 68-86%. From these disclosures the present inventors believe the COX inhibitors will reach their target at therapeutically effective concentrations.

The invention further comprises the compositions defined above in relation to the first aspect of their invention, for use in the treatment of a further indication, namely malignant tumours, by embolisation with release of the active at the site of embolisation.

The embolic compositions may be administered in the normal manner for tumour embolisation. Thus the composition may be admixed immediately before administration by the interventional radiologist, with imaging agents such as radiopaque agents. Alternatively or additionally, the particles may be preloaded with radiopaque material in addition to the pharmaceutical active. Thus the polymer and pharmaceutical active, provided in preformed admixture, may be mixed with a radiopaque imaging agent in a syringe, used as the reservoir for the delivery device. The composition may be administered, for instance, from a microcatheter device, into the appropriate artery. Selection of suitable particle size range, dependent upon the desired site of embolisation may be made in the normal way by the interventional radiologists.

The example is illustrated in the following examples and figures, in which

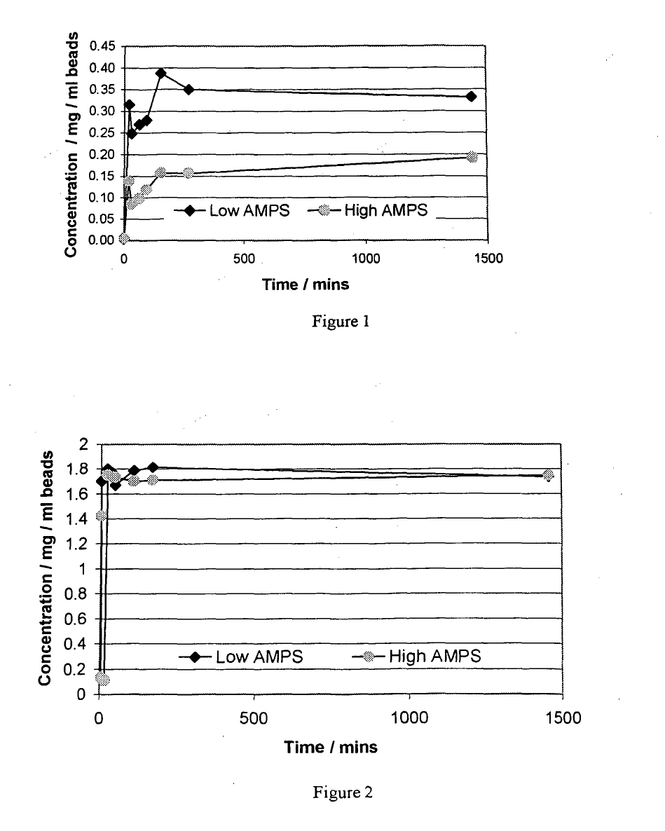

FIG. 1 shows the results of the loading described in example 2 of ibuprofen from PBS;

FIG. 2 shows the results of the loading of example 2 using ibuprofen in ethanol;

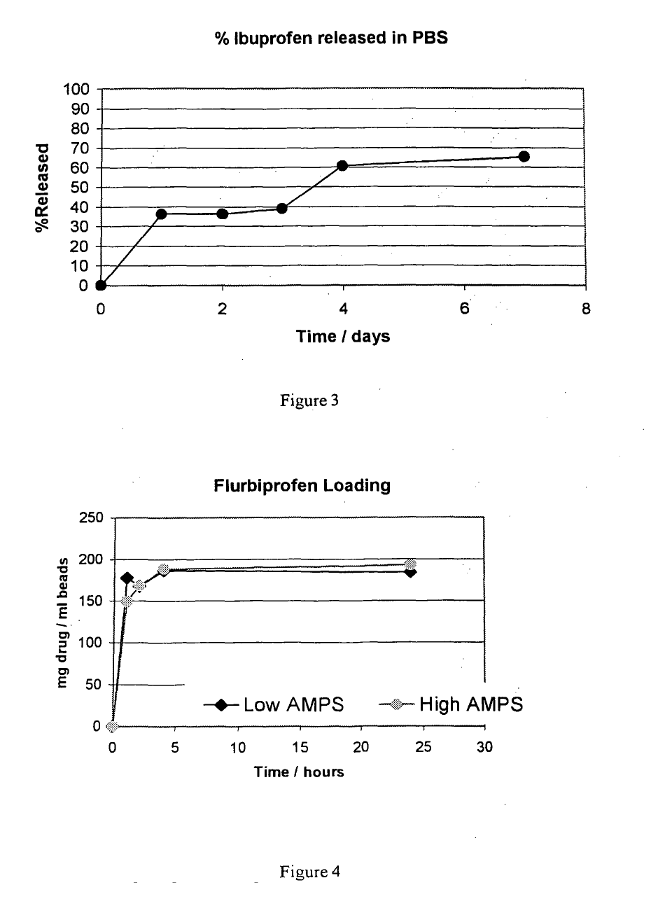

FIG. 3 shows the release profile of ibuprofen (loaded from ethanol) into PBS from the low AMPS product in example 2;

FIG. 4 shows the loading of profile of Flurbiprofen in low and high AMPS beads of example 3;

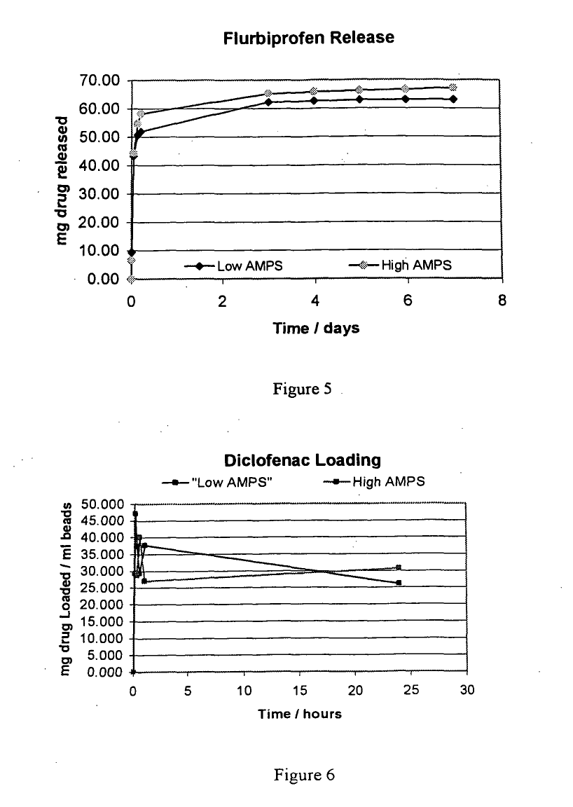

FIG. 5 shows the release of Flurbiprofen from beads low and high AMPS beads of example 3;

FIG. 6 shows the loading of Diclofenac in low and high AMPS beads of example 4;

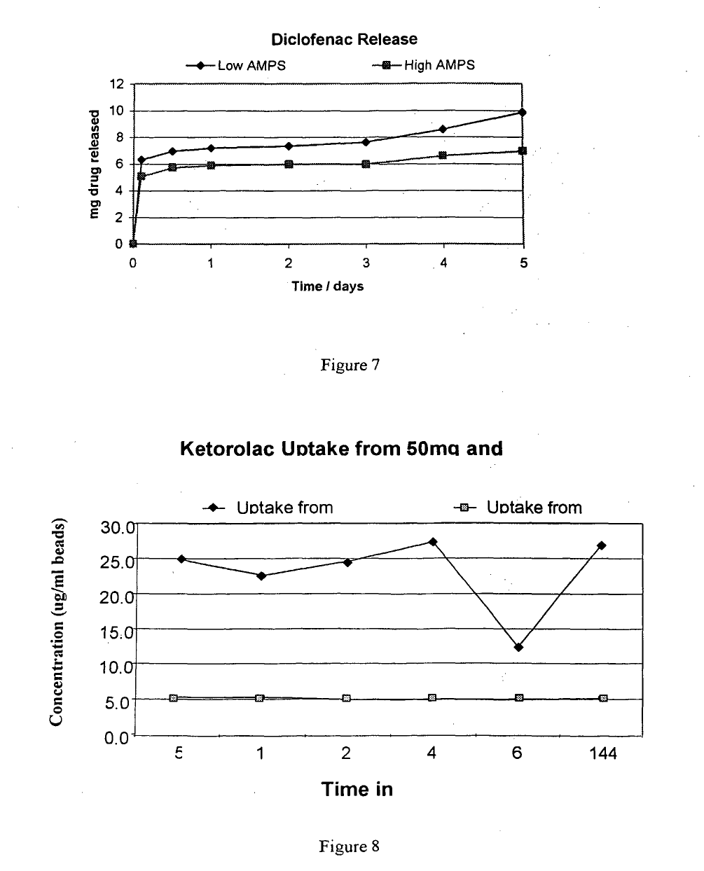

FIG. 7 shows the release of Diclofenac from beads of the present invention of example 4;

FIG. 8 shows the ketorolac loading in low AMPS microspheres of example 5;

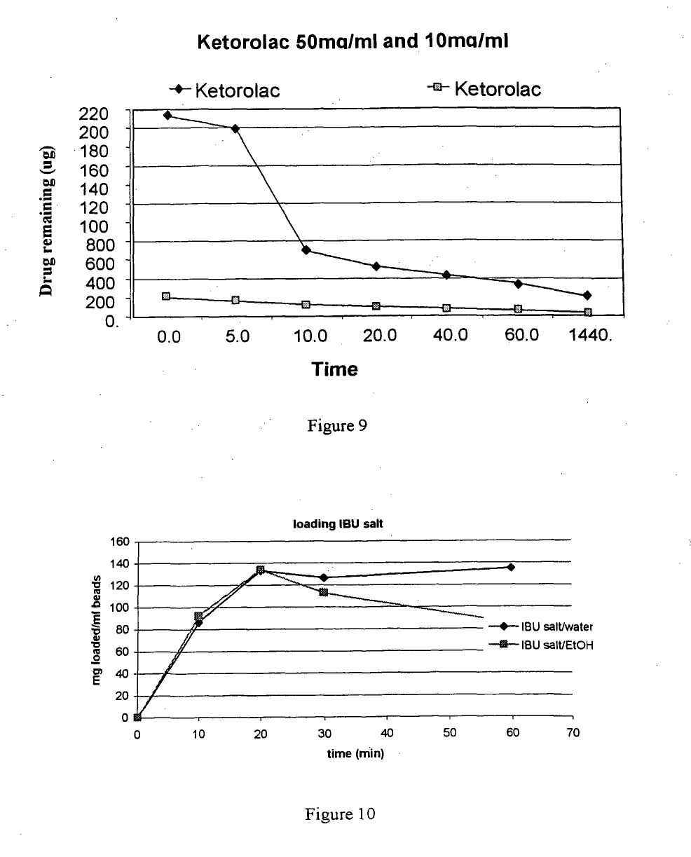

FIG. 9 shows the release of ketorolac from low AMPS microspheres of example 5;

FIG. 10 shows the loading of ibuprofen sodium salt from microspheres of example 7;

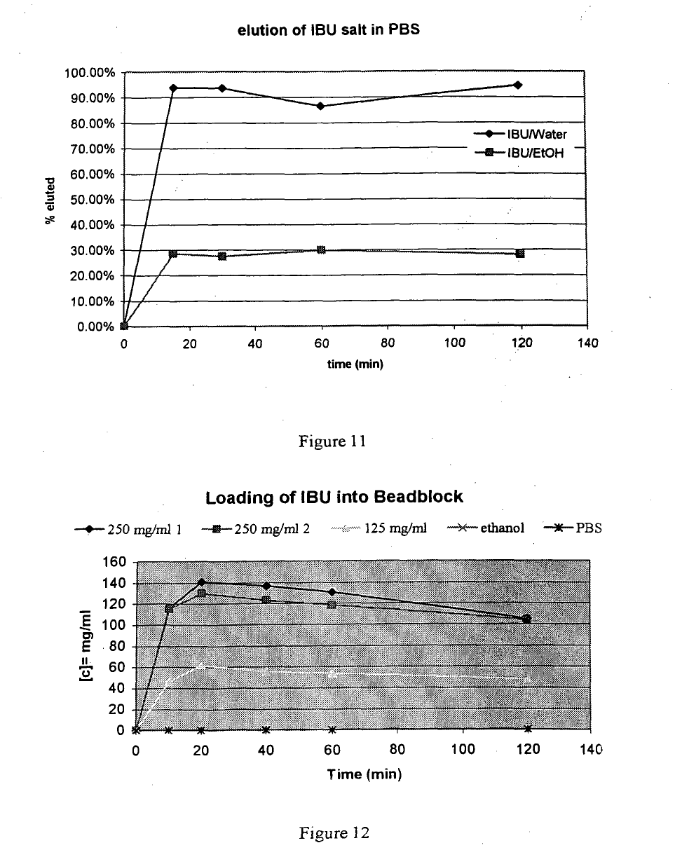

FIG. 11 shows the release of ibuprofen sodium salt from microspheres of example 7;

FIG. 12 shows the loading of ibuprofen free acid into microspheres of example 8;

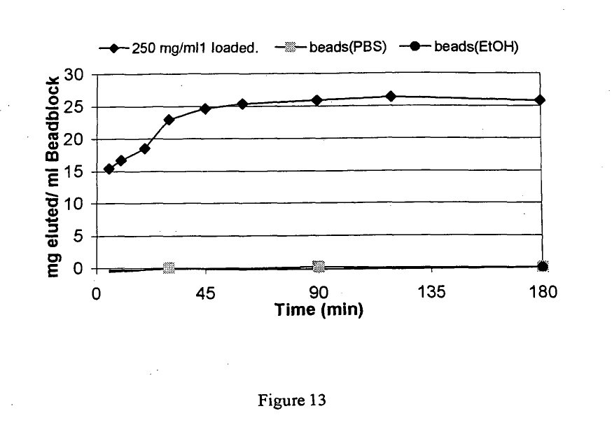

FIG. 13 shows the release of ibuprofen free acid from microspheres of example 8;

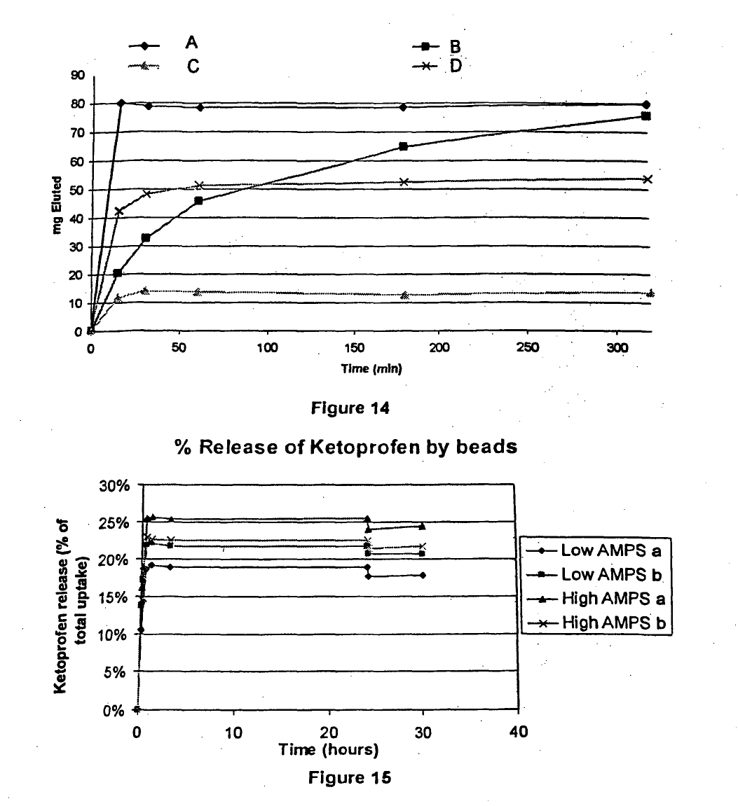

FIG. 14 shows the release of ibuprofen into PBS from microspheres loaded under different conditions of example 9;

FIG. 15 shows the release of ketoprofen from beads of the present is invention of example 10;

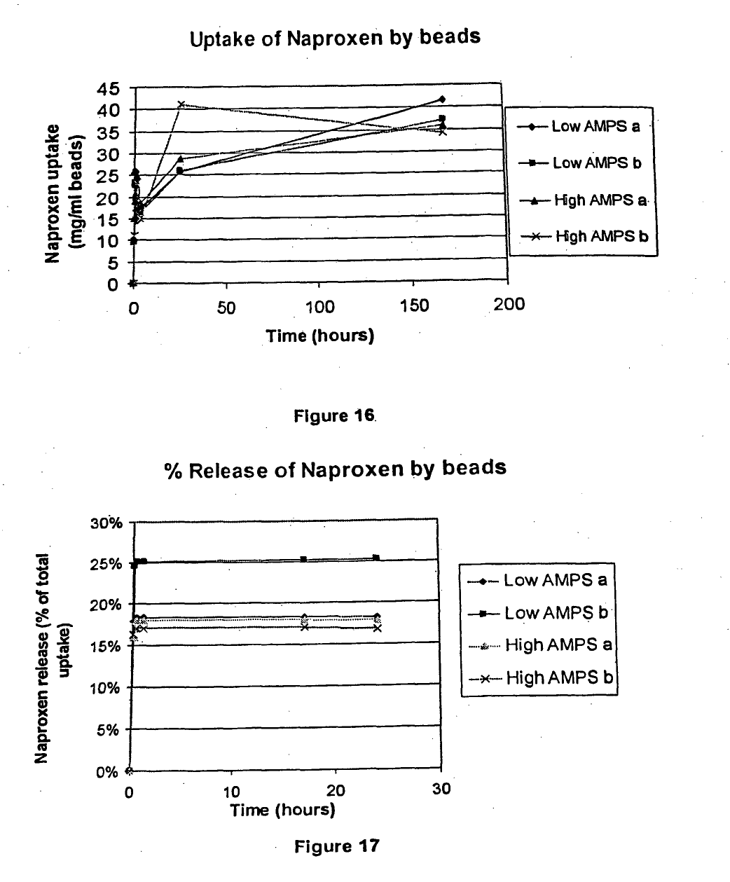

FIG. 16 shows the uptake of naproxen by microspheres of example 11;

FIG. 17 shows the release of naproxen from microspheres of example 11;

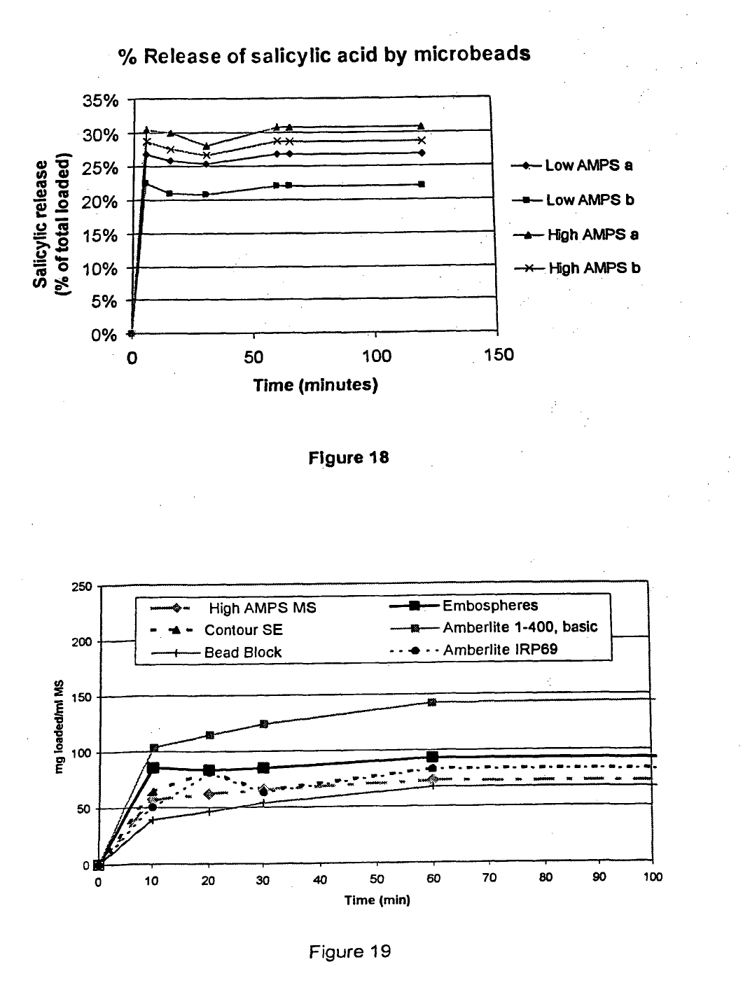

FIG. 18 shows the release of salicylic acid from microspheres of example 12;

FIG. 19 shows the loading rates of various microspheres with ibuprofen as in Example 13;

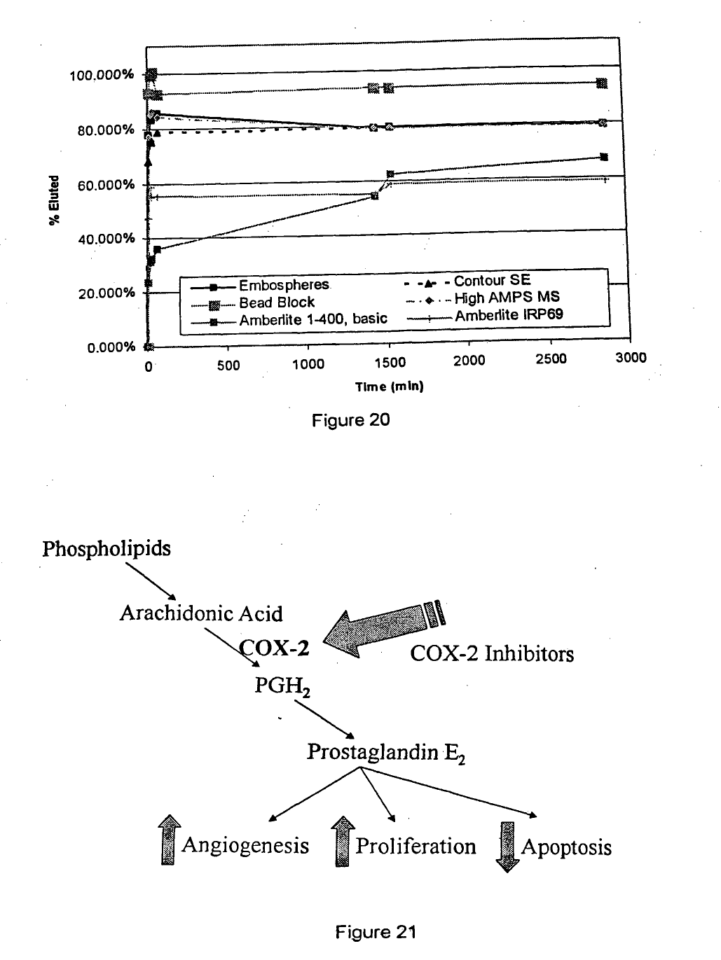

FIG. 20 shows the release rates of ibuprofen from microspheres as in Example 13;

FIG. 21 is a schematic diagram of the role of COX-2 inhibition in controlling tumourigenesis;

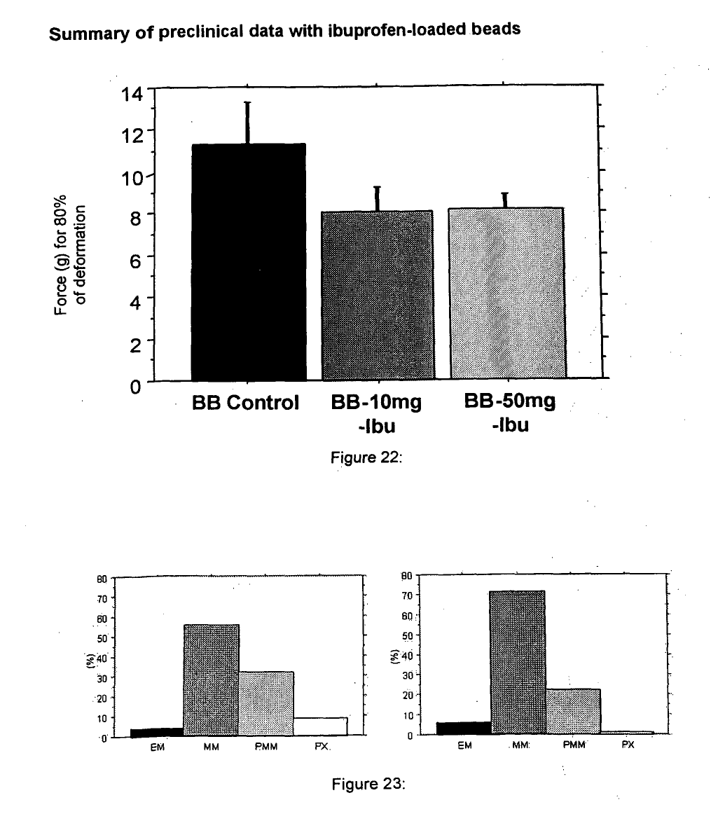

FIG. 22 shows the results for Example 14.1.1;

FIG. 23 shows the results for Example 14.1.2.

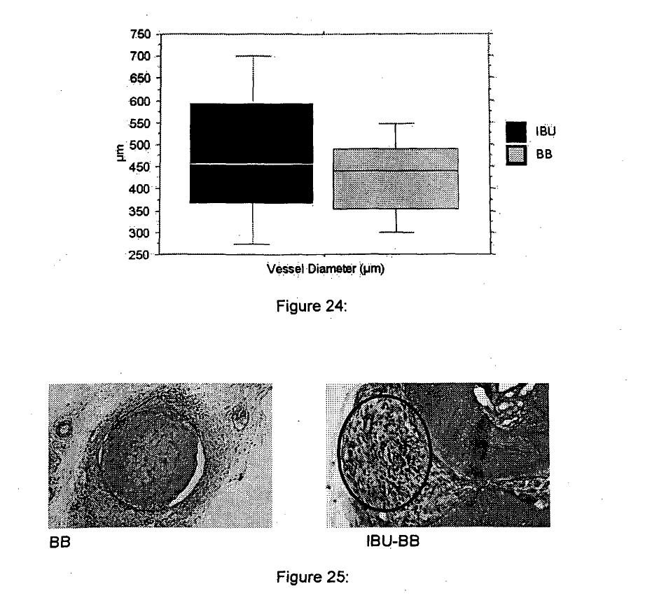

FIG. 24 shows the results for Example 14.1.2;

FIG. 25 shows the results for Example 14.2.1;

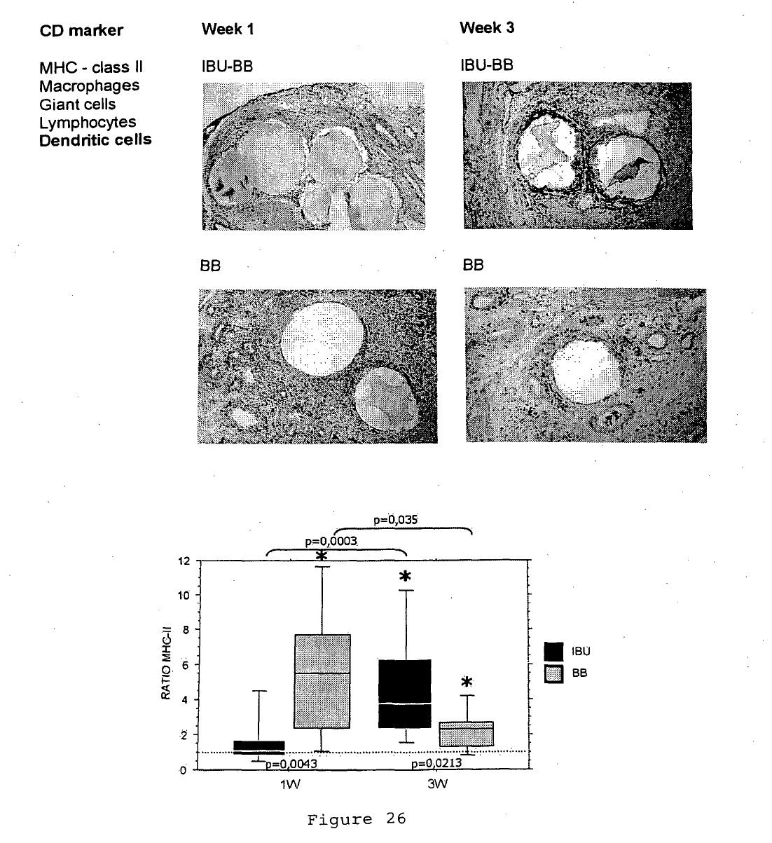

FIG. 26 shows the results for Example 14.2.2;

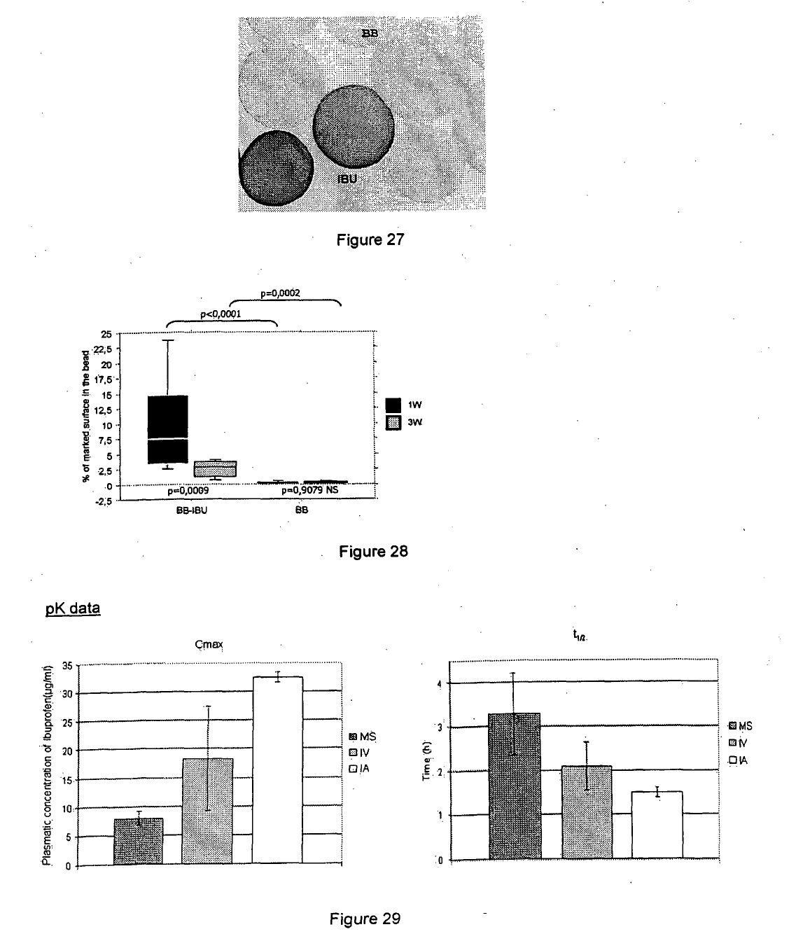

FIG. 27 shows the results for Example 14.2.3;

FIG. 28 shows the results for Example 14.2.3; and

FIG. 29 shows the results for Example 14.2.4.

### EXAMPLE 1

### Outline Method for the Preparation of Microspheres

Nelfilcon B Macromer Synthesis:

The first stage of microsphere synthesis involves the preparation of Nelfilcon B—a polymerisable macromer from the widely used water soluble polymer PVA. Mowiol 8-88 poly(vinyl alcohol) (PVA) powder (88% hydrolised, 12% acetate content, average molecular weight about 67,000 D) (150 g) (Clariant, Charlotte, N.C. USA) is added to a 2 l glass reaction vessel. With gentle stirring, 1000 ml water is added and the stirring increased to 400 rpm. To ensure complete dissolution of the PVA, the temperature is raised to 99±9° C. for 2-3 hours. On cooling to room temperature N-acryloylaminoacetaldehyde (NAAADA) (Ciba Vision, Germany) (2.49 g or 0.104 mmol/g of PVA) is mixed in to the PVA solution followed by the addition of concentrated hydrochloric acid (100 ml) which catalyses the addition of the NAAADA to the PVA by transesterification. The reaction proceeds at room temperature for 6-7 hours then stopped by neutralisation to pH 7.4 using 2.5M sodium hydroxide solution. The resulting sodium chloride plus any unreacted NAAADA is removed by diafiltration (step 2).

Diafiltration of Macromer:

Diafiltration (tangential flow filtration) works by continuously circulating a feed solution to be purified (in this case nelfilcon B solution) across the surface of a membrane allowing the permeation of unwanted material (NaCl, NAAADA) which goes to waste whilst having a pore size small enough to prevent the passage of the retentate which remains in circulation.

Nelfilcon B diafiltration is performed using a stainless steel Pellicon 2 Mini holder stacked with 0.1 m² cellulose membranes having a pore size with a molecular weight cut off of 3000 (Millipore Corporation, Bedford, Mass. USA). Mowiol 8-88 has a weight average molecular weight of 67000 and therefore has limited ability to permeate through the membranes.

The flask containing the macromer is furnished with a magnetic stirrer bar and placed on a stirrer plate. The solution is fed in to the diafiltration assembly via a Masterflex LS peristaltic pump fitted with an Easy Load II pump head and using LS24 class VI tubing. The Nelfilcon is circulated over the membranes at approximately 50 psi to accelerate permeation. When the solution has been concentrated to about 1000 ml the volume is kept constant by the addition of water at the same rate that the filtrate is being collected to waste until 6000 ml extra has been added. Once achieved, the solution is concentrated to 20-23% solids with a viscosity of 1700-3400 cP at 25° C. Nelfilcon is characterised by GFC, NMR and viscosity.

Microsphere Synthesis:

The spheres are synthesised by a method of suspension polymerisation in which an aqueous phase (nelfilcon B) is added to an organic phase (butyl acetate) where the phases are immiscible. By employing rapid mixing the aqueous phase can be dispersed to form droplets, the size and stability of which can be controlled by factors such as stirring rates, viscosity, ratio of aqueous/organic phase and the use of stabilisers and surfactants which influence the interfacial energy between the phases. Two series of microspheres are manufactured, a low AMPS and a higher AMPS series, the formulation of which are shown below.

| A | High AMPS: |
|---|---|
| Aqueous: | ca 21% w/w Nelfilcon B solution (400 ± 50 g approx) |
| | ca 50% w/w 2-acrylamido-2-methylpropanesulphonate Na salt |
| | (140 ± 10 g) |
| | Purified water (137 ± 30 g) |
| | Potassium persulphate (5.22 ± 0.1 g) |
| | Tetramethyl ethylene diamine TMEDA (6.4 ± 0.1 ml) |
| Organic: | n-Butyl acetate (2.7 ± 0.3 L) |
| | 10% w/w cellulose acetate butyrate in ethyl acetate |
| | (46 ± 0.5 g) |
| | Purified water (19.0 ± 0.5 ml) |
| B | Low AMPS: |
| Aqueous: | ca 21% w/w Nelfilcon B solution (900 ± 100 g approx) |
| | ca 50% w/w 2-acryamido-2-methylpropanesulphonate Na salt |
| | (30.6 ± 6 g) |
| | Purified water (426 ± 80 g) |
| | Potassium persulphate (20.88 ± 0.2 g) |
| | TMEDA (25.6 ± 0.5 ml) |
| Organic: | n-Butyl acetate (2.2 ± 0.3 L) |
| | 10% w/w cellulose acetate butyrate (CAB) in ethyl acetate |
| | (92 ± 1.0 g) |
| | Purified water (16.7 ± 0.5 ml) |

A jacketed 4000 ml reaction vessel is heated using a computer controlled bath (Julabo PN 9-300-650) with feedback sensors continually monitoring the reaction temperature.

The butyl acetate is added to the reactor at 25° C. followed by the CAB solution and water. The system is purged with nitrogen for 15 minutes before the PVA macromer is added. Crosslinking of the dispersed PVA solution is initiated by the addition of TMEDA and raising the temperature to 55° C. for three hours under nitrogen. Crosslinking occurs via a redox initiated polymerisation whereby the amino groups of the TMEDA react with the peroxide group of the potassium persulphate to generate radical species. These radicals then initiate polymerisation and crosslinking of the double bonds on the PVA and AMPS transforming the dispersed PVA-AMPS droplets into insoluble polymer microspheres. After cooling to 25° C. the product is transferred to a filter reactor for purification where the butyl acetate is removed by filtration followed by:
Wash with 2×300 ml ethyl acetate to remove butyl acetate and CAB
Equilibrate in ethyl acetate for 30 mins then filtered
Wash with 2×300 ml ethyl acetate under vacuum filtration
Equilibrate in acetone for 30 mins and filter to remove ethyl acetate, CAB and water
Wash with 2×300 ml acetone under vacuum filtration
Equilibrate in acetone overnight
Wash with 2×300 ml acetone under vacuum
Vacuum dry, 2 hrs, 55° C. to remove residual solvents.

Dyeing:

This step is optional but generally unnecessary when drug is loaded with a coloured active (as this provides the colour). When hydrated the microsphere contains about 90% (w/w) water and can be difficult to visualise. To aid visualisation in a clinical setting the spheres are dyed blue using reactive blue #4 dye (RB4). RB4 is a water soluble chlorotriazine dye which under alkaline conditions will react with the pendant hydroxyl groups on the PVA backbone generating a covalent ether linkage. The reaction is carried out at pH 12 (NaOH) whereby the generated HCl will be neutralised resulting in NaCl.

Prior to dyeing, the spheres are fully re-hydrated and divided into 35 g aliquots (treated individually). Dye solution is prepared by dissolving 0.8 g RB4 in 2.5M NaOH solution (25 ml) and water (15 ml) then adding to the spheres in 2 l of 80 g/l⁻¹ saline. After mixing for 20 mins the product is collected on a 32 μm sieve and rinsed to remove the bulk of the unreacted dye.

Extraction:

An extensive extraction process is used to remove any unbound or non specifically adsorbed RB4. The protocol followed is as shown:
Equilibrate in 2 l water for 5 mins. Collect on sieve and rinse. Repeat 5 times
Equilibrate in 2 l solution of 80 mM disodium hydrogen. phosphate in 0.29% (w/w) saline. Heat to boiling for 30 mins. Cool, collect on sieve and wash with 1 l saline. Repeat twice more.
Collect, wash on sieve the equilibrate in 2 l water for 10 mins.
Collect and dehydrate in 1 l acetone for 30 mins.
Combine all aliquots and equilibrate overnight in 2 l acetone.

Sieving:

The manufactured microsphere product ranges in size from 100 to 1200 microns and must undergo fractionation through a sieving process using a range of mesh sizes to obtain the nominal distributions listed below.

| | | |
|---|---|---|
| 1. | 100-300 | μm |
| 2. | 300-500 | μm |
| 3. | 500-700 | μm |
| 4. | 700-900 | μm |
| 5. | 900-1200 | μm |

Prior to sieving, the spheres are vacuum dried to remove any solvent then equilibrated at 60° C. in water to fully re-hydrate. The spheres are sieved using a 316 L stainless steel vortisieve unit (MM Industries, Salem Ohio) with 38 cm (15 in) stainless steel sieving trays with mesh sizes ranging from 32 to 1000 μm. Filtered saline is recirculated through the unit to aid fractionation. Spheres collected in the 32 micron sieve are discarded.

### EXAMPLE 2

### Uptake and Elution of Ibuprofen in Low AMPS and High AMPS Microspheres

Two solutions were prepared, one 2.5 mg per ml of ibuprofen (in phosphate buffer solution), the second 2.5 mg per ml in ethanol. Standard curves of both solutions were measured by UV absorption at 250 nm. The standard curves were used to monitor the uptake of drug by the microspheres.

For each of the Low AMPS and High AMPS microspheres four 1 ml syringes were filled with 0.25 ml of microspheres. Two glass vials were charged with 5 ml of the 2.5 mg/ml drug in PBS and a further two vials with 5 ml of PBS to act as controls. This was repeated for the drug in ethanol and two control vials of 5 ml of ethanol, again for controls. Taking two of the Low AMPS microsphere filled syringes, the contents of one was added to the vial containing drug solution in PBS and the second syringe added to its equivalent control vial. This was repeated for two of the High AMPS microsphere filled syringes. The whole process was then repeated with the ethanol solutions.

Uptake of ibuprofen was monitored using 1 ml of solution, replaced each time to keep the concentration constant, by UV spectrometry at 250 nm. The resulting absorbencies were used to calculate the amount of drug loaded in mg per ml of microspheres.

Absorbance (solution)−Absorbance of control=Actual Absorbance of drug loaded.

Concentration was calculated using the relevant standard curve and converted to give the concentration of drug which could be loaded into 1 ml of microspheres.

The results of the uptake from PBS over a period of one day are shown in FIG. 1. The results of the uptake from ethanol are shown in FIG. 2.

Release of ibuprofen from the ethanol loaded low AMPS microspheres were made in 5 ml PBS and monitored over 7 days. Concentrations were calculated using the PBS standard curve. The results are shown in FIG. 3 which shows the percentage of the total released over the 7 day period.

### EXAMPLE 3

### Loading and Release of Flurbiprofen from Microspheres

A solution of 100 mg/ml flurbiprofen (Sigma) in ethanol was prepared. 5 ml of the solution was added to 0.5 ml of microspheres/beads of the present invention, made as outlined in example 1. Low AMPS and high AMPS microspheres of size 500-710 μm were used and drug uptake monitored by UV. The samples were agitated on a roller mixer. Aliquots of supernatant were taken at 10, 20, 30, 60 mins and then at 2 hr, out to 24 hr. Uptake was calculated from the flurbiprofen remaining in solution. Both types of the microspheres were loaded with similar doses of 195 mg (low AMPS) and 197 (high AMPS bead) per ml of hydrated microspheres (FIG. 4), and in less than 30 minutes, 99% of the drug solution is located in the microspheres. Microspheres of the present invention of each size loaded with 200 mg/ml flurbiprofen were placed in 250 ml water at 37° C. 30% release was achieved in first 10 minutes with a further 5% in 2 days. If microspheres were transferred to 100 ml of elutant, release was slow until eventually equilibrium was reached (FIG. 5).

### EXAMPLE 4

### Loading and Release of Diclofenac from Microspheres

A solution of 100 mg/ml diclofenac (Sigma) in ethanol was prepared. 5 ml of the solution was added to 0.5 ml of low AMPS and high AMPS microspheres of the present invention produced as outlined in example 1; both samples used microspheres having size range 500-710 μm, and uptake monitored by UV. The samples were agitated on a roller mnixer. Aliquots of supernatant were taken at 5, 15, 30 and 240 mins and then 24 hr. Uptake was calculated from the diclofenac remaining in solution. Both types of the microspheres were loaded with similar doses of 26 mg (low AMPS beads) and 30 mg (high AMPS beads) per ml of hydrated microspheres (FIG. 6), and in less than 30 minutes, 99% of the drug solution is located in the microspheres. Microspheres of the present invention of each size loaded with 26 and 30 mg/ml diclofenac were placed in 250 ml water at 37° C. 18-26% release in first 5 minutes with a further 35% in 48 hrs (FIG. 7).

### EXAMPLE 5

### Loading and Release of Ketorolac from Microspheres

Two solutions of 50 mg/ml and 10 mg/ml ketorolac (Sigma) in water were prepared. 5 ml of the solution was added to 0.5 ml of low AMPS microspheres, of size 500-710 μm, and uptake monitored by HPLC. The samples were agitated on a roller mixer. Aliquots of supernatant were taken at 5, 10, 20 40 and 60 mins and then 24 hr. Uptake was calculated from the ketorolac remaining in solution. The microspheres were loaded with similar approximately doses half the concentrations of the original loading solutions per ml of hydrated microspheres (FIG. 8), and in less than 10 minutes, 99% of the drug solution is located in the microspheres. Microspheres of each type loaded with 13 mg and 27 mg/ml ketorolac were placed in 250 ml water at 37° C. From the high AMPS loaded microspheres 43% released in first 5 minutes with a 90% in 1 hrs this was followed with a slow release of a further 4% in the next 24 hrs (FIG. 9). The low loaded microspheres showed a similar profile with a higher amount of ketorolac 75% released in first 5 minutes, 90% in 1 hr and a further 5% in next 24 hrs.

### EXAMPLE 6

### Loading and Release of Ibuprofen Free Acid from Microspheres

A series of experiments were carried out, using a loading solution containing 250 mg/ml solution of Ibuprofen free acid (Sigma) in ethanol (Romil). 2 ml of this solutions was added to 1 ml of hydrated low AMPS microspheres made as described in example 1, and uptake monitored by UV of the supernatant at 263 nm. The samples were agitated on a roller mixer. Samples of the supernatant were taken at 10, 20, 40, 60 mins and 24 hrs. Uptake was calculated from the ibuprofen remaining in solution. The microspheres could be loaded with different doses ranging from to 142-335 mg per ml of hydrated microspheres. Elution experiments were carried out on these microspheres (table 1). Microspheres were washed to determine quick burst in various media as in table 1. Then samples were placed in 10 ml solvent and absorbance read after 10 mins, a further 20 ml added and absorbance read after 10 mins, this was repeated up to 90 mls and elution was monitored up to 24 hrs (table 1). Elution rate ranged between 20%-43% with an average of 25% in most experiments and approximately 15% was quick burst.

TABLE 1
Elution experiments of Ibuprofen Free Acid

| Loading | Loading | Eluted | Quick | |
|---|---|---|---|---|
| solution | mg/ml | Drug | Burst/Wash | Elution Solvent |
| ml | Bead | (mg) | out Solvent | Used |
| 2 | 187.08 | 47 | 100% ethanol | 50% ethanol |
| 2 | 207.7 | 53 | 50% ethanol | 50% ethanol |
| 2 | 235.53 | 60 | 100% ethanol | 0.9% Saline |
| | | | | (pH 12) |
| 2 | 177.3 | 47 | 0.9% Saline | 0.9% Saline |
| | | | (pH 12) | (pH 12) |
| 2 | 185.24 | 83 | 0.9% Saline | 0.9% Saline |
| | | | (pH 12) | (pH 12) |
| 2 | 142.82 | 57 | 0.9% Saline | 0.9% Saline |
| | | | (pH 12) | (pH 12) |
| 3 | 323.7 | 77 | 0.9% Saline | 0.9% Saline |
| | | | (pH 12) | (pH 12) |

### EXAMPLE 7

### Loading of Release of Ibuprofen Sodium Salt from Microspheres

Two samples of 1 ml of hydrated Low AMPS beads (700-1100 μm, example 1) were used. For preparation of the loading solutions: a) 1 g of ibuprofen sodium salt (SIGMA) was dissolved in 4 ml of water (ROMIL) and b) 1 g of ibuprofen sodium salt (SIGMA) was dissolved in 4 ml of ethanol (ROMIL) to give a final concentration of 250 mg/ml. Once prepared, the absorbances of the solutions were read by UV at 263 nm and dilutions were made to produce a standard curve. 2 ml of the Ibuprofen solution was added to a vial containing 1 ml of beads and timing was started. The vials were placed on a roller mixer at room temperature for the entire experiment. At a predetermined time points (0, 10, 20, 30 and 60 min) 100 μl was removed, diluted as necessary (1/200) and read at 263 nm. From the readings and the standard curve, the concentration of the solution at each time point was calculated. The amount of drug loaded onto the beads was measured by the depletion of the drug in solution when extracted with the beads. From the data the mg drug loaded per 1 ml of hydrated beads were calculated and the graph plotted. From the data shown in FIG. 10 it can be seen that when the ibuprofen is loaded from ethanol a maximum loading is reached in about 20 minutes before loading levels again begin to decrease. This is a consequence of a competition between drug/solvent penetration into the microspheres and a concomitant de-swelling of the beads as the ethanol dehydrates them. After 20 minutes the de-swelling becomes predominant and some of the drug solution is forced from the interstices of the bead as its structure collapses.

For elution studies, 1 ml of the 250 mg/ml loaded beads was transferred into a glass-brown container filled with 100 ml of PBS and timing was started. The containers were placed in the roller mixer at room temperature for the entire experiment. At predetermined times (15, 30, 60 and 120 minutes) 1 ml of the solution was removed, read and then placed is back into the container, so the volume remained constant for the entire experiment. Samples were read at 263 nm and concentrations were calculated from the equation of the ibuprofen standard curve. From the data, the mg of drug eluted per 1 ml of hydrated beads was calculated and the graph plotted (FIG. 11).

### EXAMPLE 8

### Loading and Elution of Ibuprofen Free Acid from Microspheres

Five samples of 1 ml of hydrated beads Low AMPS 700 to 1100 μm were used. For each sample, 1 ml of beads in phosphate buffered saline (PBS), measured with a 10 ml—glass cylinder, was transferred to a glass container and all the PBS was carefully removed with a glass Pasteur pipette. For preparing the loading solutions: 2 g of Ibuprofen free acid (SIGMA) was dissolved in 8 ml of ethanol (ROMIL) to give a final concentration of 250 mg/ml. Once prepared, the absorbances of the solution and dilutions were read by UV at 263 nm to produce a standard curve. 2 ml of the ibuprofen solution was added to a vial containing 1 ml of beads (previously prepared, details above) and timing was started. This was done in duplicate; in the second experiment 1 ml of ibuprofen solution was added to 1 ml of ethanol (so the final concentration of the solution was 125 mg/ml). As controls 2 ml of ethanol was added to one vial and 2 ml of PBS was added to another vial, each vial containing 1 ml of beads. The vials were placed on the roller mixer at room temperature for the entire experiment. At predetermined time points (0, 20, 40, 60 and 120 min) 100 μl was removed, diluted as necessary (1/200) and read at 263 nm. From the readings and the standard curve, the concentration of the solution at each time point was calculated. The amount of drug loaded onto the beads was measured by the depletion of the drug in solution. From the data the mg drug loaded per 1 ml of beads were calculated and the graph plotted (FIG. 12). Again, as in example 7, the contraction of the beads when exposed to ethanol causes an optimum loading to be obtained at around 20 mins before contraction causes expulsion of the drug solution from the beads.

Loaded beads from the experiment above were used for elution experiments. 1 ml of the 250 mg/ml loaded beads was transferred into a glass-brown container filled with 20 ml of PBS and timing was started. The containers were placed in the roller mixer at room temperature for the entire experiment. At time 10 minutes, 30 ml of fresh PBS was added into the container and at time 2 h another 50 ml of PBS was added into the container to give a final volume of 100 ml. At predetermined time points (0, 5, 10, 20, 30, 45, 60, 90 min and 2, 3 and 24 hours) 1 ml of the solution was removed, read and then placed back into the container. Samples were read at 263 nm and concentrations were calculated from the equation of the ibuprofen standard curve. From the data, the mg of drug eluted per 1 ml of hydrated beads was calculated and the graph plotted (FIG. 13). Controls from the experiment above were eluted in the same conditions.

### EXAMPLE 9

### Loading and Elution of Ibuprofen into Microspheres using pH and Solvent Triggers

Six samples of 1 ml of beads (700-1100 μm) were used. For each sample, 1 ml of beads in phosphate buffered saline (PBS), measured with a 10 ml glass cylinder, was transferred to a glass container and all the PBS was carefully removed with a glass Pasteur pipette. For preparing the loading solutions: a) 4 g of ibuprofen sodium salt (SIGMA) were dissolved in 16 ml of water (ROMIL) to give a final concentration of 250 mg/ml and b) 1 g of ibuprofen free acid (SIGMA) was dissolved in 4 ml of ethanol (ROMIL) to give a final concentration of 250 mg/ml. Once prepared, the absorbances of the solution and dilutions of the aqueous and of the alcoholic solutions were read by UV at 263 nm to produce standard curves. The aqueous loading solution of ibuprofen sodium salt was then used to load 3 samples (A, B and C) of beads. Sample A was loaded by adding 2 ml of the ibuprofen salt solution to a vial containing 1 ml of hydrated beads for 20 minutes (previously prepared, details above). The vial was placed on the roller mixer at room temperature for the entire experiment. Once loaded, the remaining solution was removed, measured in a graduated measurement cylinder and read at 263 nm. From the readings and the standard curve, the concentration of the solution was calculated. The amount of drug loaded onto the beads was calculated by the subtracting the amount of drug in solution from the amount in the starting loading solution. From the data the mg drug loaded per 1 ml of beads for sample A was 101 mg/ml. As a control 2 ml water with no drug was “loaded” into beads.

For sample B, the loading was the same as for sample A, but, instead of the residual liquid being immediates removed, 2 ml of water at pH 1 (obtained by adding HCl to the water) was added to the vial. This was kept in the roller mixer for 20 minutes. After that, the solution was removed, and the concentration of ibuprofen remaining was determined and thus the amount loaded into the beads. The loading for sample B was found to be 129.5 mg/ml loading. As control 2 ml of water at pH 1 was added to a vial containing 1 ml of beads.

For sample C 2 ml of ethanol for 20 min; after that, the solution was removed and the concentration or ibuprofen free acid remaining was determined thereby allowign calculation of the amount loaded into the bead. The amount loaded was found to be 47 mg/ml bead. As control, for sample C, 2 ml of ethanol was added to a vial containing 1 ml of beads.

In sample D, 2 ml of the ethanol solution containing 250 mg/ml of ibuprofen free acid was added and kept in the roller mixer for 20 minutes. After that, the solution was removed and the concentration of ibuprofen determined. The loading of ibuprofen free acid in to the bead was found to be 110.8 mg/ml.

Elution was carried out with 1 ml of the loaded beads transferred into a glass-brown container filled with 100 ml of PBS and timing was started. The containers were placed in the roller mixer at room temperature for the entire experiment. At predetermined times (15, 30, 60 and 3 and 5 hours) 1 ml of the solution was removed, read and then placed back into the container, so the volume remained constant for the entire experiment. Samples were read at 263 nm and concentrations were calculated from the equation of the ibuprofen standard curve. From the data, the amount of drug eluted per 1 ml of hydrated beads was calculated and the graph plotted (FIG. 14). Controls from the experiment above were eluted in the same conditions. Controls are not presented in the graphs because the concentrations eluted remained below detection limits from the entire experiment.

It can be seen that where the pH has been adjusted, release of the ibuprofen is slowed significantly. This is due to the generation of the ibuprofen free acid in-situ within the beads and hence the solubility of the drug is drastically decreased. Similarly, if the beads are exposed to ethanol after loading, the structure is collapsed due to water expulsion (as in Example 7). Upon rehydration in the buffer, the release profile of the free acid is slowed even more, suggesting that the collapsing process helps to impede drug dissolution from the polymer matrix.

### EXAMPLE 10

### Loading and Release of Ketoprofen from Microspheres

A ketoprofen solution of 30 mg/ml in ethanol was prepared (Sigma Aldrich). 0.5 ml of 500-710 μm low AMPS or high AMPS type microspheres (example 1) was added to 5 ml of ketoprofen solution in duplicate (a & b), and uptake was monitored by UV over 72 hours. After an initially higher uptake which was not maintained, maximum loading occurred at 24 hours with the low AMPS microspheres showing approximately 12 mg ketoprofen loaded/ml spheres and the high AMPS microspheres showing approximately 10 mg ketoprofen loaded/ml spheres.

Release of ketoprofen from the spheres loaded for 24 hours was determined as follows: the excess loading solution was removed by glass Pasteur pipette from the loaded microspheres described above. Each sample of loaded microspheres was placed in a glass jar containing 100 ml water and the jars were placed in a shaking water bath at 37° C. Release was measured by UV over 24 hours, at which point a further 100 ml water was added to each jar. UV measurement was continued for 6 hours after this. Approximately 20-25% of the loaded drug was released from the microspheres, this being equivalent to approximately 2.5 mg/ml of microspheres. (% calculated from the maximum loading obtained after 24 hours). This was released in the first 15 minutes of the elution. The addition of extra water after 24 hours did not bring about any further release of the drug (FIG. 15). There appeared to be little effect on release rate between the low and high AMPS in the microsphere formulation.

### EXAMPLE 11

### Loading and Release of Naproxen from Microspheres

A naproxen solution of 30 mg/ml in ethanol was prepared from naproxen obtained from Sigma Aldrich. 0.5 ml of 500-710 μm low AMPS or high AMPS microspheres was added to 5 ml of naproxen solution in duplicate, and uptake was monitored by UV over 168 hours (7 days). The microspheres took up approximately 35-40 mg naproxen/ml of spheres over 168 hours. Initial rapid uptake was followed by apparent partial release, then more gradual uptake (FIG. 16).

The excess loading solution was removed by glass Pasteur pipette from the loaded microspheres described in Example 8. Each sample of loaded microspheres was placed in a glass vial containing 10 ml water and the vials were placed in a shaking water bath at 37° C. Release was measured by UV over 17 hours, at which point the microspheres were placed in 10 ml fresh water. UV measurement were continued for 7 hours after this. Approximately 17-25% of the loaded drug was released from the microspheres, this being equivalent to approximately 6-9 mg/ml of microspheres. This was released in the first 5 minutes of the elution (FIG. 17). The transfer of the microspheres to fresh water after 17 hours did not bring about any further release of the drug.

### EXAMPLE 12

### Loading and Release of Salicylic acid from Microspheres

A salicylic acid solution of 5 mg/ml in ethanol was prepared from salicylic acid obtained from Sigma Aldrich. 0.5 ml of 500-710 μm low AMPS or high AMPS microspheres were added to 5 ml of salicylic acid solution in duplicate, and uptake was monitored by UV over 24 hours. The microspheres took up a maximum of approximately 3-4 mg salicylic acid/ml of microspheres after 3-4 hours, but this had decreased to 2-3 mg/ml of microspheres after 24 hours.

The elution of the drug was assessed as follows: the excess loading solution was removed by glass Pasteur pipette from the loaded microspheres. Each sample of loaded microspheres was placed in a glass jar containing 100 ml water and the vials were placed in a shaking water bath at 37° C. Release was measured by UV over 60 hours, at which point the microspheres were placed in 10 ml fresh water. UV measurement were continued for 60 hours after this. The low AMPS microspheres released approximately 25% of the salicylic acid loaded, whereas the high AMPS microspheres released approximately 30% of the salicylic acid loaded. For both microsphere types the majority of the drug was released within the first 15 minutes (FIG. 18). The transferral of the spheres into fresh water did not bring about any further release of the drug.

### EXAMPLE 13

### Loading and Elution of Ibuprofen in Different Microspherical Agents

The following microsphere products were tested:
1. High AMPS microsphere (made as in Example 1) particle size fraction 595-710 μm, equilibrium water content 94%.
2. Contour SE, a commercially available embolic product comprising non-ionic polyvinylalcohol microspheres particle size fraction 500-700 μm, equilibrium water content 40%.
3. Low AMPS microspheres made as in Example 1 above particle size range 500 to 700 μm, equilibrium water content 90%.
4. Embosphere—a commercially available embolic agent comprising particles of N-acryloyl-2-amino-2-hydroxy methyl-propane-1,3-diol-co-N,N-bisacrylamide) copolymer cross-linked with gelatin and glutaraldehyde having particle size range 500 to 700 μm. This polymer at neutral pH has a net positive charge from the gelatin content. (FR-A-7723223). The equilibrium water content is 91%.
5. Amberlite I-400, a basic ion-exchange material formed from quaternary amino-functionalised styrene DVB copolymer, particle size 230 to 810 μm (average 512 μm), equilibrium water content at 37° C. in distilled water 52%.
6. Amberlite IRP69—an acidic ion-exchange medium formed from sulphonic acid—functionalised styrene—DVB copolymer (dry particle size 25 to 150 μm) equilibrium water content 57%.

1 ml of hydrated microspheres were loaded using a 2 ml volume of 100 mg/ml concentration of ibuprofen sodium salt in water. Loading levels were checked over a 100 minute period and found to vary between 67 to 142 mg of drug per ml of hydrated beads. The results are shown in FIG. 19. The basic Amberlite resin was seen to load more drug which is a consequence of the interaction between the positive charges on the resin and the negative carboxylate of the drug.

Elution of 2 ml of loaded beads was performed in 100 ml of PBS over a 2 hour period;. The resuls are shown in FIG. 20. Elution of the drug is rapid due to its high water solubility, except in the case of the positively charged Amberlite resin where the charge interaction slowed the release from the spheres.

### EXAMPLE 14

### 14.1 Product Related Performance Data

14.1.1 Compression and Elasticity

The purpose of this study was to evaluate the impact of Ibuprofen loading on rigidity and elasticity of microspheres. Low Amps microsphere produced as in Example 1 above 900-1200 μm unloaded (BB), or loaded with 10 or 50 mg of ibuprofen (IBU-BB) the different levels achieved by adjusting the concentration of drug in the loading solution, as well as unloaded micrrospheres that have been through the same process as the loaded beads were tested.

Compression:

Compression or a single microsphere was analysed using a Texture Analyser (TA-XTPlus Micro Stable Systems, Vienna). The aim was to measure the force required to compress at 10 micron s⁻¹ from 10 to 80% reduction from the starting diameter. FIG. 22 shows a comparison of the force for 80% deformation of microspheres; M-W test p=0.009 BB control vs IBU-BB.

These data indicate that the control is more rigid at a compression of 80%, than ibuprofen-loaded microsphere product. However at lower compression there appears to be no significant difference.

Stress Recovery:

The speed of recovery was measured after a compression of 40% at 10 micron s⁻¹ for microsphere product and product loaded with ibuprofen, by immediate removal of the stress and monitoring the recovery by optical camera. There was no significant difference in the speed of recovery, therefore the elasticity, of the different microspheres.

14.1.2 Localisation

The purpose of this study is to evaluate the distribution of microspheres (Low Amps made according to the process in Example 1 500-700 μm) and ibuprofen-loaded (100 mg) microspheres within the sheep uterus. An angiographic evaluation was performed to assess level occluded and the extent of arterial occlusion in each organ. Histological analysis was used to determine the localization of beads within the different artery sizes of the two organs, as well as assess the local tissue reaction to the products.

Results indicate a significant difference in the localisation of the IBU-BB, with the IBU-BB microspheres occluding the vessels more proximally than the BB microspheres. FIG. 23 shows a localisation of microspheres after uterine artery embolisation in a sheep model p=0.0014 X². EM: endometrium; MM: myometrium; PMM: perimyometrium; PX: proximal.

In addition, the mean vessel diameter in which the IBU-BB microspheres were located was larger than the vessels in which the BB microspheres were located. FIG. 24 shows the mean vessel diameter of IBU-BB microspheres and BB microspheres p=0.0044 (MW).

### 14.2 Biological Activity

The purpose of this study was to assess the anti-inflammatory effect of IBU-BB over a 3-week period post-embolisation of the uterine artery of sheep.

14.2.1 Inflammation

Haematoxylin and eosin stained sections of uterus were examined for the main cell types present at week 1 and week 3 post-embolisation. A semi-quantitative analysis indicates that there are less lymphocytes at 1 week after embolisation with IBU-BB than with BB. However, the reverse is observed at week 3, i.e. there were more lymphocytes embolisation with IBU-BB than with BB. FIG. 25 shows presence of lymphocytes at week 3 post emobilisation with either BB or IBU-BB. No significant difference was seen in the levels of neutrophils or eosinophils.

14.2.2 CD Markers

The analysis of CD markers present on the lymphocytes was completed by quantify the relative amount of marked surfaces in the embolized area compared to control area. The quantification confirmed the delayed inflammatory reaction with BB-IBU noted above. An example for MHC class II labelling is shown in FIG. 26. FIG. 26 shows quantification of MHC class II at week 1 and week 3. Statistical analysis was a univariate test vs. value 1. The other markers that showed a similar pattern were CD172a, CD3 and CD4. Of interest, no CD8 marking was observed with either BB product, whereas the presence of CD8 positive cells has been observed with other products. This is a marker of cytotoxicity and a good measure of biocompatibility of biomaterials.

14.2.3 Antibody Staining for IBU

Specific staining of IBU-BB was detected using an anti-ibuprofen polyclonal antibody. FIG. 27 shows staining of BB and IBU with ibuprofen-specific polyclonal antibody. The amount (surface area stained) of IBU detected in the beads was around 8% at 1 week and 2.5% at week 3. FIG. 28 shows analysis of the amount (surface area stained) of Ibuprofen detected on the beads at week 1 and week 3.

14.2.4 PharmacoKinetic Data

Methods: An in vivo study of the plasma levels of ibuprofen after administration by uterine artery embolisation with IBU-BB (MS), inter-uterine administration of ibuprofen solution (IA), or administration in to the jugular vein of ibuprofen solution (IV).
MS study (100 mg/ml, 500-700 μm): intra-uterine injection of 0.5 ml of MS in the left artery and 0.5 ml into the right one, n=5
IV study: injection of 65 mg solution into the jugular vein, n=3
IA study: injection of 50 mg solution in both the left and right artery, n=3

The plasmatic concentrations were measured. FIG. 29 shows Cₘₐₓ (left panel) and t_{1/2} (right panel) calculated from the plasmatic levels of ibuprofen after embolisation (MS), intra-arterial (IA) or intravenous (IV) administration. FIG. 29 indicates that the highest level after embolisation with MS were approximately 7 μg/ml compared to 32 μg/ml after intra-arterial administration. In addition, the increased t_{1/2} of MS compared to IA indicates a longer presence in the body of the ibuprofen after embolisation.

### 14.3 Conclusions

The results demonstrate that ibuprofen is released locally into the arterial wall and surrounding tissue by the presence of the difference in inflammatory cell populations and CD markers. Hence, it is releasing and having an effect on cells, thus it is reasonable to assume it may have an effect on tumour cells as well.

Studies carried out to show ibuprofen has an effect on tumour volume (e.g. Yao et al, Clin Cancer Res, 11, 1618-1628, 2005—effects of non-selective COX inhibition with low-dose ibuprofen on tumour growth, angiogenesis, metastasis and survival in a mouse model of colorectal cancer). The drug is given orally, not locally. This reference shows that the drug has an effect on tumourogenesis. The above results show that the drug delivery is locally at a dose that has a biological effect in-vivo.

The elasticity/rigidity data show that although the embolic contains a drug, it still maintains the important physical characteristics of an embolisation bead—that it is compressible down a microcatheter and that the bead recovers from the deformation, so that the location of embolisation can be predicted.

1-20. (canceled)

21. A method of treatment of an animal suffering from a malignant tumor comprising the step of administering a composition comprising a water-insoluble polymer and, associated with the polymer in a releasable form, a pharmaceutically active agent which is a COX inhibitor, whereby the tumor is embolized and the pharmaceutical active is released from the polymer at the site of embolization.

22. Method of treatment according to claim 21, in which the polymer is in the form of particles.

23. Method of treatment according to claim 22, in which the particles are substantially spherical in shape.

24. Method of treatment according to claim 22, in which the particles have particle sizes when equilibrated in water at 37° C. in the range 40 to 1500 μm.

25. Method of treatment according to claim 21, in which the particles are water-swellable.

26. Method of treatment according to claim 21, in which the COX inhibitor is selective for COX-1.

27. Method of treatment according to claim 21, in which the COX inhibitor is selective for COX-2.

28. Method of treatment according to claim 21, in which the pharmaceutically active agent is selected from the group consisting of celecoxib, rofecoxib, diclofenac, diflunisal, etodolac, flurbiprofen, ibuprofen, indomethacin, ketoprofen, ketorolac, nabumetone, naproxen, oxaprozin, piroxicam, sulindac, and tolmetin and pharmaceutically acceptable salts thereof.

29. Method of treatment according to claim 21, in which the pharmaceutically active agent is selected from the group consisting of ibuprofen, flurbiprofen, diclofenac, ketorolac, naproxen, ketoprofen and salicyclic acid and pharmaceutically acceptable salts thereof.

30. Method of treatment according to claim 21, in which the pharmaceutical active is present in the composition at a concentration in the range 0.1 to 1000 mg/ml.

31. Method of treatment according to claim 21, in which the polymer is synthetic and biostable.

32. Method of treatment according to claim 21, in which the polymer is cross-linked.

33. Method of treatment according to claim 32, in which the polymer is covalently cross-linked.

34. Method of treatment according to claim 21, in which the polymer is formed by the radical polymerization of poly(vinyl alcohol) macromer having pendant ethylenically unsaturated groups.

35. Method of treatment according to claim 34, in which the pendant groups are (alk) acrylic groups.

36. Method of treatment according to claim 34, in which the macromer is copolymerized with ethylenically unsaturated comonomer.

37. Method of treatment according to claim 36, in which the comonomer is ionic comonomer.

38. Method of treatment according to claim 36, in which the comonomer is an acrylic compound.

39. A composition comprising a water-insoluble polymer and, associated with the polymer in a releasable form, a pharmaceutically active agent which is a COX inhibitor, for use in a method of malignant tumor embolization, in which the pharmaceutical active is released from the polymer at the site of embolization.

40. A composition according to claim 39, in which the pharmaceutically active agent is selected from the group consisting of celecoxib, rofecoxib, diclofenac, diflunisal, etodolac, flurbiprofen, ibuprofen, indomethacin, ketoprofen, ketorolac, nabumetone, naproxen, oxaprozin, piroxicam, sulindac, and tolmetin and pharmaceutically acceptable salts thereof.

41. A composition according to claim 39, in which the pharmaceutically active agent is selected from the group consisting of ibuprofen, flurbiprofen, diclofenac, ketorolac, naproxen, ketoprofen and salicyclic acid and pharmaceutically acceptable salts thereof.

42. A composition according to claim 39, in which the polymer is in the form of substantially spherical water-swellable particles having particle sizes when equilibrated in water at 37□C in the range 100 to 1200 μm and is a synthetic, biostable, covalently cross-linked polymer.

43. A composition according to claim 42, in which the polymer is formed by the radical polymerization of poly(vinyl alcohol) macromer having pendant (alk) acrylic groups copolymerized with ethylenically unsaturated comonomer.
